SEQUENCE LISTING (24 sequences)

SEQ ID NO: 1 - DNA, 2072 bp, Artificial Sequence
  Human factor VII active site mutant immunoconjugate mRNA, complete coding sequence
aagctttgcagagatttcatcatggtctcccaggccctcaggctcctctgccttctgcttgggcttcagggctgcctggctgcagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgaagctgttctggatttcttacagtgatggtgaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaagccccaagggcgaattgtggggggcaaggtgtgccccaaaggggagtgtccatggcaggtcctgttgttggtgaatggagctcagttgtgtggggggaccctgatcaacaccatctgggtggtctccgcggcccactgtttcgacaaaatcaagaactggaggaacctgatcgcggtgctcggggagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcgcgggggacagtggaggcccacatgccacccactaccggggcacgtggtacctgacgggcatcgtcagctggggccagggctgcgcaaccgtgggccactttggggtgtacaccagggtctcccagtacatcgagtggctgcaaaagctcatgcgctcagagccacgcccaggagtcctcctgcgagccccatttcccggatccgcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataagcggccgc

SEQ ID NO: 2 - protein, 641 aa, Artificial Sequence
  Human factor VII mutant immunoconjugate AA sequence
  MOD_RES (6)..(6): May be glutamic acid or a post translationally modified gamma carboxy-glutamic acid (GLA)
  MOD_RES (7)..(7): May be glutamic acid or a post translationally modified gamma carboxy-glutamic acid (GLA)
  MOD_RES (14)..(14): May be glutamic acid or a post translationally modified gamma carboxy-glutamic acid (GLA)
  MOD_RES (16)..(16): May be glutamic acid or a post translationally modified gamma carboxy-glutamic acid (GLA)
  MOD_RES (19)..(19): May be glutamic acid or a post translationally modified gamma carboxy-glutamic acid (GLA)
  MOD_RES (20)..(20): May be glutamic acid or a post translationally modified gamma carboxy-glutamic acid (GLA)
  MOD_RES (25)..(25): May be glutamic acid or a post translationally modified gamma carboxy-glutamic acid (GLA)
  MOD_RES (26)..(26): May be glutamic acid or a post translationally modified gamma carboxy-glutamic acid (GLA)
  MOD_RES (29)..(29): May be glutamic acid or a post translationally modified gamma carboxy-glutamic acid (GLA)
  MOD_RES (34)..(34): May be glutamic acid or a post translationally modified gamma carboxy-glutamic acid (GLA)
  CARBOHYD (52)..(52): May be site of O-glycosylation
  CARBOHYD (60)..(60): May be site of O-glycosylation
  CARBOHYD (145)..(145): May be site of N-glycosylation
  CARBOHYD (322)..(322): May be site of N-glycosylation
  CARBOHYD (491)..(491): May be site of N-glycosylation
ANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCAGDSGGPHATHYRGTWYLTGIVSWGQGCATVGHFGVYTRVSQYIEWLQKLMRSEPRPGVLLRAPFPGSAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 641 aa, Artificial Sequence
  Human factor VII mutant immunoconjugate AA sequence
  MOD_RES (6)..(6): May be glutamic acid or a post translationally modified gamma carboxy-glutamic acid (GLA)
  MOD_RES (7)..(7): May be glutamic acid or a post translationally modified gamma carboxy-glutamic acid (GLA)
  MOD_RES (14)..(14): May be glutamic acid or a post translationally modified gamma carboxy-glutamic acid (GLA)
  MOD_RES (16)..(16): May be glutamic acid or a post translationally modified gamma carboxy-glutamic acid (GLA)
  MOD_RES (19)..(19): May be glutamic acid or a post translationally modified gamma carboxy-glutamic acid (GLA)
  MOD_RES (20)..(20): May be glutamic acid or a post translationally modified gamma carboxy-glutamic acid (GLA)
  MOD_RES (25)..(25): May be glutamic acid or a post translationally modified gamma carboxy-glutamic acid (GLA)
  MOD_RES (26)..(26): May be glutamic acid or a post translationally modified gamma carboxy-glutamic acid (GLA)
  MOD_RES (29)..(29): May be glutamic acid or a post translationally modified gamma carboxy-glutamic acid (GLA)
  MOD_RES (34)..(34): May be glutamic acid or a post translationally modified gamma carboxy-glutamic acid (GLA)
  CARBOHYD (52)..(52): May be site of O-glycosylation
  CARBOHYD (60)..(60): May be site of O-glycosylation
  CARBOHYD (145)..(145): May be site of N-glycosylation
  CARBOHYD (322)..(322): May be site of N-glycosylation
  CARBOHYD (491)..(491): May be site of N-glycosylation
ANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDAGGPHATHYRGTWYLTGIVSWGQGCATVGHFGVYTRVSQYIEWLQKLMRSEPRPGVLLRAPFPGSAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - DNA, 2057 bp, Artificial Sequence
  Coding sequence for human factor VII mutant immunoconjugate AA sequence
ggatccaccatggtgtcacaagccctccgcctgctctgcctccttctcggacttcaaggatgtctggccgcagtgttcgtgactcaagaagaggcccacggggtgctgcaccggaggcgccgcgcgaacgcctttctggaggagctgcggccaggctcgctggaaagagagtgcaaagaggagcaatgctccttcgaggaggccagagaaattttcaaggacgccgaacggactaagctgttttggatctcatacagcgacggcgaccagtgtgccagcagcccgtgccagaacggcggttcgtgcaaggaccagctgcagtcctacatttgtttctgcctgccggcgttcgaaggaagaaactgtgaaacccacaaggatgaccagctgatctgcgtgaacgagaacggaggctgcgaacagtactgctccgaccacacggggactaagaggagctgcagatgccatgagggctatagcctgctggccgacggggtgtcctgcactccgactgtggaatacccgtgcggaaagatccccatcctggagaagcggaacgcatccaagccccaaggacgaattgtcggcggaaaggtctgccctaagggagagtgtccttggcaagtgctcctcttggtgaacggagcgcagctttgcggcggaactctgatcaacaccatctgggtggtgtcggcagctcactgctttgataagattaagaactggcggaacctgatcgcggtgcttggcgaacatgacctgtccgaacacgacggggacgagcagtcccggagagtggcccaggtcatcattccatccacctacgtgccggggaccacaaaccacgacatcgccttgctgcggctgcaccagccggtggtccttaccgatcacgtcgtgccgctctgcctgcccgagcgcacctttagcgagcgcaccctggccttcgtgcgcttctccctggtgtccggctggggacagctgctggacagaggcgccaccgccctggaactgatggtgctgaacgtgccacggctcatgacccaagattgtctgcagcagtcgcgcaaagtcggggattcaccgaacatcaccgagtacatgttctgcgccggttactccgatggatcgaaggactcgtgcgctggagactccggtggaccccatgctacccattatcgcggtacttggtacctgactggtattgtgtcctggggacagggctgcgccaccgtgggacatttcggcgtgtacacaagggtgtcacagtacatcgaatggttgcagaagctcatgcgcagcgaacctcgcccgggagtgctcctgagggcccctttccccggaagcgctgagcctaagagctgcgacaagacccacacttgtccgccatgccccgcgcccgaactgctgggtggcccatccgtgttcctgttcccgcctaagcccaaggacaccctgatgatttcgcggacccccgaagtgacttgcgtggtggtggacgtcagccacgaagatccggaagtcaagttcaattggtacgtggatggcgtcgaagtccataacgccaagaccaagccccgcgaggagcagtacaattccacttaccgggtggtgtccgtgttgaccgtgttgcaccaggactggctgaacggaaaggaatacaagtgcaaagtgtcgaataaggccctcccggccccgatcgagaaaacgatctccaaggccaagggccagcctcgggagccccaggtctacaccttgccgccgtcccgggatgagctgaccaagaaccaggtgtcactcacctgtctcgtcaaggggttctacccttccgacatcgccgtggaatgggagtcgaacgggcagccggaaaacaattacaagaccactcctcctgtcctggattccgacgggtcattcttcctgtactcaaagctgaccgtggacaagagcagatggcaacagggcaacgtgttcagctgctccgtgatgcacgaggccctgcacaaccactacacccagaagtccctgtccctctctcccggaaaatgagcggccgc

SEQ ID NO: 5 - DNA, 2057 bp, Artificial Sequence
  Coding sequence for human factor VII mutant immunoconjugate AA sequence
tctagaccaccatggtgtcacaagccctccgcctgctctgcctccttctcggacttcaaggatgtctggccgcagtgttcgtgactcaagaagaggcccacggggtgctgcaccggaggcgccgcgcgaacgcctttctggaggagctgcggccaggctcgctggaaagagagtgcaaagaggagcaatgctccttcgaggaggccagagaaattttcaaggacgccgaacggactaagctgttttggatctcatacagcgacggcgaccagtgtgccagcagcccgtgccagaacggcggttcgtgcaaggaccagctgcagtcctacatttgtttctgcctgccggcgttcgaaggaagaaactgtgaaacccacaaggatgaccagctgatctgcgtgaacgagaacggaggctgcgaacagtactgctccgaccacacggggactaagaggagctgcagatgccatgagggctatagcctgctggccgacggggtgtcctgcactccgactgtggaatacccgtgcggaaagatccccatcctggagaagcggaacgcatccaagccccaaggacgaattgtcggcggaaaggtctgccctaagggagagtgtccttggcaagtgctcctcttggtgaacggagcgcagctttgcggcggaactctgatcaacaccatctgggtggtgtcggcagctcactgctttgataagattaagaactggcggaacctgatcgcggtgcttggcgaacatgacctgtccgaacacgacggggacgagcagtcccggagagtggcccaggtcatcattccatccacctacgtgccggggaccacaaaccacgacatcgccttgctgcggctgcaccagccggtggtccttaccgatcacgtcgtgccgctctgcctgcccgagcgcacctttagcgagcgcaccctggccttcgtgcgcttctccctggtgtccggctggggacagctgctggacagaggcgccaccgccctggaactgatggtgctgaacgtgccacggctcatgacccaagattgtctgcagcagtcgcgcaaagtcggggattcaccgaacatcaccgagtacatgttctgcgccggttactccgatggatcgaaggactcgtgcgctggagactccggtggaccccatgctacccattatcgcggtacttggtacctgactggtattgtgtcctggggacagggctgcgccaccgtgggacatttcggcgtgtacacaagggtgtcacagtacatcgaatggttgcagaagctcatgcgcagcgaacctcgcccgggagtgctcctgagggcccctttccccggaagcgctgagcctaagagctgcgacaagacccacacttgtccgccatgccccgcgcccgaactgctgggtggcccatccgtgttcctgttcccgcctaagcccaaggacaccctgatgatttcgcggacccccgaagtgacttgcgtggtggtggacgtcagccacgaagatccggaagtcaagttcaattggtacgtggatggcgtcgaagtccataacgccaagaccaagccccgcgaggagcagtacaattccacttaccgggtggtgtccgtgttgaccgtgttgcaccaggactggctgaacggaaaggaatacaagtgcaaagtgtcgaataaggccctcccggccccgatcgagaaaacgatctccaaggccaagggccagcctcgggagccccaggtctacaccttgccgccgtcccgggatgagctgaccaagaaccaggtgtcactcacctgtctcgtcaaggggttctacccttccgacatcgccgtggaatgggagtcgaacgggcagccggaaaacaattacaagaccactcctcctgtcctggattccgacgggtcattcttcctgtactcaaagctgaccgtggacaagagcagatggcaacagggcaacgtgttcagctgctccgtgatgcacgaggccctgcacaaccactacacccagaagtccctgtccctctctcccggaaaatgaaccggt

SEQ ID NO: 6 - protein, 29 aa, Artificial Sequence
  Hinge Region
GVLLRAPFPGSAEPKSCDKTHTCPPCPAP

SEQ ID NO: 7 - protein, 29 aa, Artificial Sequence
  Mutated Hinge/Linker Region
GVLLRAPFPGSAEPKSSDKTHTCPPCPAP

SEQ ID NO: 8 - protein, 29 aa, Artificial Sequence
  Mutated Hinge/Linker Region
GVLLRAPFPGSAEPASSDKTHTCPPCPAP

SEQ ID NO: 9 - protein, 24 aa, Artificial Sequence
  Mutated Hinge Region
GVLLRAPFPGSADKTHTCPPCPAP

SEQ ID NO: 10 - protein, 25 aa, Artificial Sequence
  Mutated Hinge Region
GVLLRAPFPGGSSDKTHTCPPCPAP

SEQ ID NO: 11 - protein, 641 aa, Artificial Sequence
  Immunocongugate comprising mutated hinge region
ANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCAGDSGGPHATHYRGTWYLTGIVSWGQGCATVGHFGVYTRVSQYIEWLQKLMRSEPRPGVLLRAPFPGSAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 641 aa, Artificial Sequence
  Immunoconjugate comprising mutated hinge region
ANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCAGDSGGPHATHYRGTWYLTGIVSWGQGCATVGHFGVYTRVSQYIEWLQKLMRSEPRPGVLLRAPFPGSAEPASSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 636 aa, Artificial Sequence
  Immunocongugate comprising mutated hinge region
ANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCAGDSGGPHATHYRGTWYLTGIVSWGQGCATVGHFGVYTRVSQYIEWLQKLMRSEPRPGVLLRAPFPGSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 637 aa, Artificial Sequence
  Immunocongjugate comprising mutated hinge region
ANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCAGDSGGPHATHYRGTWYLTGIVSWGQGCATVGHFGVYTRVSQYIEWLQKLMRSEPRPGVLLRAPFPGGSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 228 aa, Artificial Sequence
  IgG Fc Region
MDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 406 aa, Artificial Sequence
  Human factor VII active site mutant
ANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCAGDSGGPHATHYRGTWYLTGIVSWGQGCATVGHFGVYTRVSQYIEWLQKLMRSEPRPGVLLRAPFP

SEQ ID NO: 17 - protein, 397 aa, Artificial Sequence
  Homo sapiens factor VII active site mutant amino acid sequence (no hinge region)
ANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCAGDSGGPHATHYRGTWYLTGIVSWGQGCATVGHFGVYTRVSQYIEWLQKLMRSEPRP

SEQ ID NO: 18 - protein, 397 aa, Artificial Sequence
  Homo sapiens factor VII active site mutant amino acid sequence (no hinge region), with mutations S344A and A341K relative to SEQ ID NO17
ANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDAGGPHATHYRGTWYLTGIVSWGQGCATVGHFGVYTRVSQYIEWLQKLMRSEPRP

SEQ ID NO: 19 - protein, 244 aa, Artificial Sequence
  Homo sapiens IgG Fc (with hinge region)
GVLLRAPFPGSAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 244 aa, Artificial Sequence
  Homo sapiens IGG Fc (with hinge region), with ProtA mutation
GVLLRAPFPGSAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 21 - protein, 244 aa, Artificial Sequence
  Homo sapiens IGG Fc (with hinge region) comprising mutated hinge region of SEQ ID NO7
GVLLRAPFPGSAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 22 - protein, 244 aa, Artificial Sequence
  Homo sapiens IgG Fc (with hinge region) comprising mutated hinge region of SEQ ID NO8
GVLLRAPFPGSAEPASSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 23 - protein, 239 aa, Artificial Sequence
  Homo sapiens IgG Fc (with hinge region) comprising mutated hinge region of SEQ ID NO9
GVLLRAPFPGSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 240 aa, Artificial Sequence
  Homo sapiens IgG Fc (with hinge region) comprising mutated hinge region of SEQ ID NO10
GVLLRAPFPGGSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNRFTQKSLSLSPGK

(19) United States
(12) Patent Application Publication
(10) Pub. No.: US 2018/0355030 A1
(43) Pub. Date: Dec. 13, 2018

(54) METHODS AND COMPOSITIONS FOR TREATING DISORDERS ASSOCIATED WITH PATHOLOGICAL NEOVASCULARIZATION

(71) Applicant: Iconic Therapeutics, Inc., South San Francisco, CA (US)

(72) Inventors: William GREENE, South San Francisco, CA (US); Kirk DORNBUSH, South San Francisco, CA (US); Thi-Sau MIGONE, South San Francisco, CA (US); Gabriela BURIAN, South San Francisco, CA (US)

(21) Appl. No.: 15/775,550

(22) PCT Filed: Nov. 12, 2016
(86) PCT No.: PCT/US16/61723

§ 371 Date: May 11, 2018

Related U.S. Application Data
(60) Provisional application No. 62/263,203, filed on Dec. 4, 2015.
(60) Provisional application No. 62/263,207, filed on Dec. 4, 2015.
(60) Provisional application No. 62/254,918, filed on Nov. 13, 2015.

(51) Int. Cl. C07K 16/22; C07K 14/745; A61K 39/395; A61K 47/64; A61P 35/00; A61P 27/02

(52) U.S. Cl. CPC C07K 16/22; C07K 14/745; A61K 39/39541; A61K 2039/505; A61P 35/00; A61P 27/02; C07K 2319/30; A61K 47/64

(57) ABSTRACT

Provided herein are immunoconjugate dimers for the treatment of disorders associated with neovascularization and tumor-associated neovascularization (e.g ocular melanoma) and symptoms associated with the same. The methods comprises administering to the patient in one or more dosing sessions, a composition comprising an effective amount of any one or more of the immunoconjugate dimers of the invention, wherein the monomer subunits of the dimer each comprises a mutated factor VIIa (FVIIa) protein conjugated to a immunoglobulin G1 (IgG1) Fc domain.

### CROSS-REFERENCE TO RELATED APPLICATIONS

This application claims the benefit of priority to U.S. Provisional Application No. 62/254,918, filed on Nov. 13, 2015, U.S. Provisional Application No. 62/263,203, filed on Dec. 4, 2015, and U.S. Provisional Application No. 62/263,207, filed on Dec. 4, 2015, each of which is hereby incorporated by reference in its entirety for all purposes.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is ICTH_005_01WO_ST25. The text file is 73 KB, was created on Nov. 11, 2016, and is being submitted electronically via EFS-Web.

### BACKGROUND OF THE INVENTION

Neovascularization (interchangeably referred to herein as angiogenesis) generally refers to the growth of existing blood vessels and the formation of new blood vessels, and is observed in a variety of diseases. Neovascularization can enable solid tumor growth and metastasis, cause visual malfunction in ocular disorders, promote leukocyte extravasation in inflammatory disorders, and/or influence the outcome of cardiovascular diseases such as atherosclerosis.

Neovascularization occurs only infrequently in healthy adults, primarily during wound healing and certain reproductive events. In contrast, neovascularization that promotes or causes disease is generally referred to as pathological neovascularization and such neovasculature is referred to as pathological neovasculature (PNV). PNV is intrinsic to several diseases and includes tumorigenic neovascularization that promotes the growth of solid cancers and melanoma, including ocular melanoma.

The survival and growth of a solid tumor depend critically on the development of a supporting neovasculature (Folkman, J. (1995) N. Engl. J. Med. 333, 1757-1763). Tumor-associated neovascularization is critical in supporting cancer progression as it supplies oxygen and nutrients. Neovascularization also plays a significant role in rheumatoid arthritis (Szekanez, Z., et al. (1998) J. Invest. Med. 46, 27-41). Neovascularization underlies the majority of eye diseases that result in catastrophic loss of vision (Friedlander, M., et al. (1996) Proc. Natl. Acad. Sci. USA 93, 9764-9769), for example in ocular melanoma and age-related macular degeneration (AMD).

Neovascularization is involved in ocular melanoma, which is a melanoma of the uveal tract, including the choroid, iris, and ciliary body. The standard of care for small- and medium-sized ocular tumors is typically radiation. Over 60% of patients receiving some form of radiation, either plaque radiotherapy (brachytherapy) or proton beam radiation. However, radiotherapies are highly invasive and can lead to complications such as retinopathy, cataracts, glaucoma, and significant vision loss. In the case of large tumors, surgical removal of the tumor or eye may be performed. None of the aforementioned treatments affect the rate at which metastatic disease occurs. While local recurrence in the eye is rare, nearly half of all uveal melanomas will develop distant metastasis, primarily in the liver.

Age-related macular degeneration (AMD) refers to the chronic, progressive degenerative pathology of the macula that results in loss of central vision. Neovascular AMD (also referred to as exudative or “wet” AMD) is the leading cause of severe vision loss and blindness in elderly patients over the age of 50 in the industrialized world.

Tissue factor (TF) is a cell surface receptor present on vascular endothelial cells. It is an integral membrane glycoprotein with an intracellular terminal domain, a transmembrane domain, and an extracellular binding domain for Factor VII (FVII) and Factor VIIa (FVIIa, activated Factor VII). TF acts as a cell-associated receptor for the activated form of coagulation Factor VII (FVIIa); the formation of this complex initiates blood coagulation and mediates cellular signaling. TF has been implicated in the process of neovascularization and the inflammatory cascade of cytokine release, both processes involved in PNV. In cancer, TF plays a significant role in multiple aspects of cancer growth in modulating tumor growth, tumor angiogenesis, metastasis, and thrombosis. In the tumor microenvironment, relative to non-transformed cells, TF is over-expressed by tumor, vascular, stromal, and some inflammatory cells.

One approach to the treatment of disorders and diseases associated with pathological neovascularization, and particularly of cancer, has been to compromise the function or growth of the neovasculature. This may be achieved by inhibiting TF, but because TF exerts multiple biological activities crucial to homeostasis, simply inhibiting TF is not a viable pharmacological solution. It is known that knocking out the TF gene in mice is lethal, and the inhibition of TF can induce hemorrhaging (Chu. Int. J. Inflam. 2011. 2011:30).

Thus there is an unmet medical need for new therapeutic strategies for treating disorders associated with neovascularization and tumor-associated neovascularization which are less invasive, provide a durable benefit, and slowing the onset or even prevent further disease progression (such as metastasis). The present invention addresses this and other needs.

Patents, patent applications, patent application publications, journal articles and protocols referenced herein are incorporated by reference in their entireties, for all purposes.

### BRIEF SUMMARY OF THE INVENTION

In one aspect of the invention, provided herein is an immunoconjugate dimer, wherein the monomer subunits of the dimer each comprises the sequence of SEQ ID NO: 11, SEQ ID NO: 12, SEQ ID NO: 13, or SEQ ID NO: 14.

In another aspect of the invention, provided herein is an immunoconjugate dimer, wherein the monomer subunits of the dimer each comprises a mutated Factor VIIa (FVIIa) targeting domain conjugated to an immunoglobulin G1 (IgG1) Fc effector domain, wherein the targeting domain comprises the sequence of SEQ ID NO: 17 or SEQ ID NO: 18, and wherein the effector domain comprises a hinge region and the effector domain comprises the sequence of SEQ ID NO:21, SEQ ID NO:22, SEQ ID NO:23, or SEQ ID NO:24.

In another aspect of the invention, provided herein is a method of treating a disease or disorder associated with neovascularization in a patient in need thereof, comprising administering to the patient in one or more dosing sessions, a composition comprising an effective amount of an immunoconjugate dimer, wherein the monomer subunits of the dimer each comprises the sequence of SEQ ID NO: 11, SEQ ID NO: 12, SEQ ID NO: 13, or SEQ ID NO:14.

In another aspect of the invention, provided herein is a method of treating a disease or disorder associated with neovascularization in a patient in need thereof, comprising administering to the patient in one or more dosing sessions, a composition comprising an effective amount of an immunoconjugate dimer, wherein the monomer subunits of the dimer each comprises a mutated Factor VIIa (FVIIa) targeting domain conjugated to an immunoglobulin G1 (IgG1) Fc effector domain, wherein the targeting domain comprises the sequence of SEQ ID NO: 17 or SEQ ID NO: 18, and wherein the effector domain comprises a hinge region and the effector domain comprises the sequence of SEQ ID NO:21, SEQ ID NO:22, SEQ ID NO:23, or SEQ ID NO:24.

In another aspect of the invention, provided herein is a method for reversing tumor neovascularization in a patient in need thereof, comprising, administering to the patient in multiple dosing sessions, a composition comprising an effective amount of the immunoconjugate dimer, wherein the monomer subunits of the dimer each comprises the sequence of SEQ ID NO: 11, SEQ ID NO: 12, SEQ ID NO: 13, or SEQ ID NO: 14; or wherein the monomer subunits of the dimer each comprises a mutated Factor VIIa (FVIIa) targeting domain conjugated to an immunoglobulin G1 (IgG1) Fc effector domain, wherein the targeting domain comprises the sequence of SEQ ID NO: 17 or SEQ ID NO: 18, and wherein the effector domain comprises a hinge region and the effector domain comprises the sequence of SEQ ID NO:21, SEQ ID NO:22, SEQ ID NO:23, or SEQ ID NO:24.

In some embodiments, the disease or disorder is atherosclerosis, rheumatoid arthritis, ocular melanoma, cancer, diabetic macular edema (DME), macular edema following retinal vein occlusion (RVO), proliferative diabetic retinopathy, wet age-related macular degeneration (AMD), retinopathy of prematurity (ROP), or neovascular glaucoma. In some embodiments, the disease or disorder is ocular melanoma. In some embodiments, the disease or disorder is wet AMD. In some embodiments, the disease or disorder is cancer.

In another aspect of the invention, provided herein is a method for treating ocular melanoma in a patient in need thereof, the method comprising administering to the patient in one or more dosing sessions, a composition comprising an effective amount of an immunoconjugate dimer, wherein the monomer subunits of the dimer each comprises a mutated Factor VIIa (FVIIa) protein conjugated to an immunoglobulin G1 (IgG1) Fc domain. In some embodiments, the mutated Factor VIIa (FVIIa) protein is human.

In another aspect of the invention, provided herein is a method for preventing or slowing metastasis of an ocular melanoma in a patient in need thereof, comprising administering to the patient in one or more dosing sessions, a composition comprising an effective amount of an immunoconjugate dimer, wherein the monomer subunits of the dimer each comprises a mutated Factor VIIa (FVIIa) protein conjugated to an immunoglobulin G1 (IgG1) Fc domain.

In another aspect of the invention, provided herein is a method for decreasing the size of an ocular melanoma tumor in an eye of a patient in need thereof, comprising, administering to the patient in one or more dosing sessions, a composition comprising an effective amount of an immunoconjugate dimer, wherein the monomer subunits of the dimer each comprises a mutated Factor VIIa (FVIIa) protein conjugated to an immunoglobulin G1 (IgG1) Fc domain.

In some embodiments, the methods further comprises administering an effective amount of a neovascularization inhibitor or an angiogenesis inhibitor to the patient.

In some embodiments, the mutated Factor VIIa (FVIIa) protein is human. In some embodiments, the immunoglobulin G1 (IgG1) Fc domain is human.

In some embodiments, treating the ocular melanoma comprises preventing, inhibiting, or reversing neovascularization in the eye of the patient in need of treatment. In some embodiments, the neovascularization is choroidal neovascularization.

In some embodiments, the tumor associated with the ocular melanoma exhibits at least a 10% reduction in size post administration of the immunoconjugate, as measured by comparing the size of the tumor having been treated versus the size of the tumor prior to treatment.

In some embodiments, the ocular melanoma is a uveal melanoma. In some embodiments, the uveal melanoma is an anterior uveal tract melanoma. In some embodiments, the anterior uveal tract melanoma is an iris melanoma. In some embodiments, the uveal melanoma is a posterior uveal tract melanoma. In some embodiments, the posterior uveal tract melanoma is a ciliary body melanoma. In some embodiments, the posterior uveal tract melanoma is a choroid melanoma.

In some embodiments, the immunoconjugate is administered in a dose of between 1 μg and 1500 μg in each of the one or more dosing sessions. In some embodiments, the dose is selected from the group consisting of about 30 μg, about 150 μg, about 300 μg, and about 600 μg. In some embodiments, the immunoconjugate is suspended in a volume of between 10 μL and 200 μL. In some embodiments, the immunoconjugate is suspended in a volume of about 20 μL, about 50 μL, or about 100 μL.

In some embodiments, the administering comprises intravitreal or suprachoroidal injection. In some embodiments, administering comprises intravenous administration. In some embodiments, administering comprises intratumoral administration. In some embodiments, treating ocular melanoma comprises slowing the onset of metastasis of the melanoma or prevention of metastasis of the melanoma in the eye of the patient in need of treatment.

In some embodiments, the immunoconjugate dimer is a homodimer. In some embodiments, the immunoconjugate dimer is a heterodimer. In some embodiments, at least one of the monomer subunits of the immunoconjugate comprises a mutated human FVIIa domain comprising a single point mutation at Lys341 or Ser344. In some embodiments, the single point mutation is to an Ala residue. In some embodiments, the single point mutation is Lys341 to Ala341. In some embodiments, the single point mutation is Ser344 to Ala344.

In some embodiments, one or more dosing sessions comprise two or more, three or more, four or more, or five or more dosing sessions. In some embodiments, each dosing session is spaced apart by from about 20 days to about 50 days, or from about 20 days to about 40 days, or from about 20 days to about 30 days. In some embodiments, there are two dosing sessions, and each dosing session is spaced apart by about 7 days. In some embodiments, said one or more dosing sessions comprise 12 to 24 dosing sessions. In some embodiments, administering comprises intravitreal injection of the composition into the eye of the patient once every 28 days, once every 30 days, or once every 35 days.

In some embodiments, the monomer subunits of the dimer each comprises a hinge region comprising SEQ ID NO: 6, 7, 8, 9 or 10. In some embodiments, at least one monomer subunit of the dimer comprises the amino acid sequence of SEQ ID NO:2, SEQ ID NO:3, SEQ ID NO:11, SEQ ID NO:12, SEQ ID NO:13, or SEQ ID NO:14. In some embodiments, at least one monomer subunit of the dimer comprises the amino acid sequence of SEQ ID NO:2. In some embodiments, at least one monomer subunit of the dimer comprises the amino acid sequence of SEQ ID NO:3. In some embodiments, at least one monomer subunit of the dimer is encoded by a polynucleotide comprising the sequence of SEQ ID NO:4. In some embodiments, at least one monomer subunit of the dimer is encoded by a polynucleotide comprising the sequence of SEQ ID NO:5. In some embodiments, the monomer subunits of the dimer each comprises a mutated Factor VIIa (FVIIa) targeting domain conjugated to an immunoglobulin G1 (IgG1) Fc effector domain, wherein the targeting domain comprises the sequence of SEQ ID NO: 17 or SEQ ID NO: 18, and wherein the effector domain comprises a hinge region and the effector domain comprises the sequence of SEQ ID NO: 19, SEQ ID NO:20, SEQ ID NO:21, SEQ ID NO:22, SEQ ID NO:23, or SEQ ID NO:24. In some embodiments, at least one monomer subunit of the dimer comprises the amino acid sequence of SEQ ID NO: 11. In some embodiments, at least one monomer subunit of the dimer comprises the amino acid sequence of SEQ ID NO: 12. In some embodiments, at least one monomer subunit of the dimer comprises the amino acid sequence of SEQ ID NO: 13. In some embodiments, at least one monomer subunit of the dimer comprises the amino acid sequence of SEQ ID NO: 14.

In some embodiments, the patient substantially maintains his or her vision subsequent to the one or more dosing sessions, as measured by losing fewer than 15 letters in a best-corrected visual acuity (BCVA) measurement, compared to the patient's BCVA measurement prior to the one or more dosing sessions. In some embodiments, the patient exhibits a decrease in the size, volume, and/or thickness of the ocular melanoma subsequent to the one or more dosing sessions, as compared to the size, volume, and/or thickness of the ocular melanoma prior to the one or more dosing sessions. In some embodiments, the decrease is measured by ultrasound, high-resolution ultrasound biomicroscopy, magnetic resonance imaging, or computed axial tomography. In some embodiments, the patient exhibits a decrease in leakage or blockage of blood vessels in the eye subsequent to the one or more dosing sessions, as compared to the leakage or blockage of blood vessels in the eye prior to the one or more dosing sessions. In some embodiments, the decrease is measured by fluorescein angiography or indocyanine green angiography. In some embodiments, the patient exhibits a decrease in swelling and/or fluid accumulation beneath the retina or choroid subsequent to the one or more dosing sessions, as compared to the swelling and/or fluid accumulation beneath the retina or choroid prior to the one or more dosing sessions. In some embodiments, the decrease is measured by ocular coherence tomography. In some embodiments, the patient exhibits a decrease in the size and/or number of iris spots subsequent to the one or more dosing sessions, as compared to the size and/or number of iris spots prior to the one or more dosing sessions. In some embodiments, the decrease is measured by a gonioscope, slit-lamp biomicroscope, or ophthalmoscope. In some embodiments, the patient experiences an improvement in vision subsequent to the one or more dosing sessions, as measured by gaining 15 letters in a best-corrected visual acuity (BCVA) measurement, compared to the patient's BCVA prior to the multiple dosing sessions. In some embodiments, subsequent to the one or more dosing sessions the neovascularization area is reduced in the eye of the patient, as compared to the neovascularization area prior to the initiation of treatment, as measured by fluorescein angiography or optical coherence tomography. In some embodiments, the patient exhibits a decrease in radiation-induced retinopathy, as compared to radiation-induced retinopathy prior to the one or more dosing sessions. In some embodiments, the decrease is due to a decreased need for radiation therapy. In some embodiments, the decrease is measured by fluorescein angiography or indocyanine green angiography.

In some embodiments, the neovascularization area is reduced by at least about 10%, at least about 20%, at least about 30%, at least about 40% or at least about 50%. In some embodiments, the decrease is by at least about 10%, at least about 20%, at least about 30%, at least about 40% or at least about 50%. In some embodiments, subsequent to the one or more dosing sessions, the retinal thickness of the eye of the patient is reduced in the eye of the patient, as compared to the retinal thickness of the eye prior to the initiation of treatment. In some embodiments, the retinal thickness is reduced by at least about 50 μm, at least about 100 μm, at least about 150 μm, at least about 175 μm, at least about 200 μm, at least about 225 μm, or at least about 250 μm. In some embodiments, the retinal thickness is reduced by at least about 10%, at least about 20%, at least about 30%, at least about 40%, or at least about 50%. In some embodiments, the decreased retinal thickness is decreased central retinal subfield thickness (CST), decreased center point thickness (CPT), or decreased central foveal thickness (CFT).

In another aspect of the invention, provided herein is a method for treating wet age-related macular degeneration (AMD) in an eye of a patient in need thereof, comprising, administering to the patient in multiple dosing sessions, a composition comprising an effective amount of the immunoconjugate dimer, wherein the monomer subunits of the dimer each comprises the sequence of SEQ ID NO: 11, SEQ ID NO: 12, SEQ ID NO: 13, or SEQ ID NO: 14. In some embodiments, treating the wet AMD comprises preventing, inhibiting or reversing choroidal neovascularization in the eye of the patient in need of treatment.

In another aspect of the invention, provided herein is a method for treating wet age-related macular degeneration (AMD) in an eye of a patient in need thereof, comprising, administering to the patient in multiple dosing sessions, a composition comprising an effective amount of the immunoconjugate dimer, wherein the monomer subunits of the dimer each comprises a mutated Factor VIIa (FVIIa) targeting domain conjugated to an immunoglobulin G1 (IgG1) Fc effector domain, wherein the targeting domain comprises the sequence of SEQ ID NO: 17 or SEQ ID NO: 18, and wherein the effector domain comprises a hinge region and the effector domain comprises the sequence of SEQ ID NO:21, SEQ ID NO:22, SEQ ID NO:23, or SEQ ID NO:24. In some embodiments, treating the wet AMD comprises preventing, inhibiting or reversing choroidal neovascularization in the eye of the patient in need of treatment.

In another aspect of the invention, provided herein is a method for preventing, inhibiting or reversing ocular neovascularization in an eye of a patient in need thereof, comprising, administering to the patient in multiple dosing sessions, a composition comprising an effective amount of the immunoconjugate dimer, wherein the monomer subunits of the dimer each comprises a mutated Factor VIIa (FVIIa) targeting domain conjugated to an immunoglobulin G1 (IgG1) Fc effector domain, wherein the targeting domain comprises the sequence of SEQ ID NO: 17 or SEQ ID NO: 18, and wherein the effector domain comprises a hinge region and the effector domain comprises the sequence of SEQ ID NO:21, SEQ ID NO:22. SEQ ID NO:23, or SEQ ID NO:24.

In some embodiments, the ocular neovascularization is associated with proliferative diabetic retinopathy, wet age-related macular degeneration (AMD), retinopathy of prematurity (ROP), or neovascular glaucoma. In some embodiments, the ocular neovascularization is secondary to proliferative diabetic retinopathy, wet age-related macular degeneration (AMD), retinopathy of prematurity (ROP), or neovascular glaucoma. In some embodiments, the ocular neovascularization is choroidal neovascularization. In some embodiments, the patient has been previously diagnosed with wet age-related macular degeneration (AMD) in the eye. In some embodiments, the choroidal neovascularization is secondary to wet AMD. In some embodiments, the eye of the patient has not been previously treated for choroidal neovascularization or wet AMD. In some embodiments, the patient has previously been treated for choroidal vascularization with anti-vascular endothelial growth factor (VEGF) therapy, laser therapy or surgery.

In some embodiments, the immunoconjugate dimer is a homodimer. In some embodiments, the immunoconjugate dimer is a heterodimer. In some embodiments, at least one of the monomer subunits of the immunoconjugate comprises a mutated human FVIIa domain comprising a single point mutation at Lys341 or Ser344. In some embodiments, the single point mutation is to an Ala residue. In some embodiments, the single point mutation is Lys341 to Ala341. In some embodiments, the single point mutation is Ser344 to Ala344.

In some embodiments, administering comprises intravitreal injection of the composition at each dosing session. In some embodiments, administering comprises suprachoroidal injection of the composition at each dosing session. In some embodiments, administering comprises intravenous administration. In some embodiments, administering comprises intratumoral injection.

In some embodiments, the multiple dosing sessions comprise two or more, three or more, four or more or five or more dosing sessions. In some embodiments, each dosing session is spaced apart by from about 20 days to about 50 days, or from about 20 days to about 40 days, or from about 20 days to about 30 days. In some embodiments, the multiple dosing sessions comprise 12 to 24 dosing sessions. In some embodiments, there are two dosing sessions, and are separated by about 7 days. In some embodiments, administering comprises intravitreal injection of the composition into the eye of the patient once every 28 days, once every 30 days or once every 35 days.

In some embodiments, the patient substantially maintains his or her vision subsequent to the multiple dosing sessions, as measured by losing fewer than 15 letters in a best-corrected visual acuity (BCVA) measurement, compared to the patient's BCVA measurement prior to the multiple dosing sessions. In some embodiments, the patient experiences an improvement in vision subsequent to the multiple dosing sessions, as measured by gaining 15 letters in a best-corrected visual acuity (BCVA) measurement, compared to the patient's BCVA prior to the multiple dosing sessions.

In some embodiments, subsequent to the multiple dosing sessions, or one or more of the dosing sessions, as measured by fluorescein angiography or optical coherence tomography, the CNV area is reduced in the eye of the patient, as compared to the CNV area prior to the initiation of treatment.

In some embodiments, the CNV area is reduced by at least about 10%, at least about 20%, at least about 30%, at least about 40% or at least about 50%. In some embodiments, subsequent to the multiple dosing sessions, or a subset thereof, the retinal thickness of the eye of the patient is reduced in the eye of the patient, as compared to the retinal thickness of the eye prior to the initiation of treatment. In some embodiments, the retinal thickness is reduced by at least about 50 μm, at least about 100 μm, at least about 150 μm, at least about 175 μm, at least about 200 μm, at least about 225 μm or at least about 250 μm. In some embodiments, the retinal thickness is reduced by at least about 10%, at least about 20%, at least about 30%, at least about 40% or at least about 50%. In some embodiments, the decreased retinal thickness is decreased central retinal subfield thickness (CST), decreased center point thickness (CPT), or decreased central foveal thickness (CFT).

In some embodiments, the method further comprises measuring the intraocular pressure (IOP) in the eye of the patient prior to each intravitreal or suprachoroidal injection. In some embodiments, the method further comprises measuring the IOP in the eye of the patient about 20 minutes, about 30 minutes, about 40 minutes, about 50 minutes or about 1 hour after each intravitreal or suprachoroidal injection. In some embodiments, the method further comprises measuring the IOP in the eye of the patient about 20 minutes, about 30 minutes, about 40 minutes, about 50 minutes or about 1 hour prior to each intravitreal or suprachoroidal injection. In some embodiments, the IOP is measured via tonometry.

In some embodiments, the method further comprises administering an effective amount of a neovascularization inhibitor or an angiogenesis inhibitor to the patient. In some embodiments, the neovascularization inhibitor or the angiogenesis inhibitor is present in the same composition as the effective amount of the immunoconjugate. In some embodiments, the neovascularization inhibitor or the angiogenesis inhibitor is present in a different composition than the effective amount of the immunoconjugate. In some embodiments, the neovascularization inhibitor is a vascular endothelial growth factor (VEGF) inhibitor, a VEGF receptor inhibitor, a platelet derived growth factor (PDGF) inhibitor or a PDGF receptor inhibitor. In some embodiments, neovascularization inhibitor is ranibizumab. In some embodiments, the dosage of ranibizumab is from about 0.2 mg to about 1 mg. In some embodiments, the dosage of ranibizumab is 0.3 mg or 0.5 mg. In some embodiments, ranibizumab is administered to the eye of the patient via an intravitreal injection. In some embodiments, the composition comprising the effective amount of the neovascularization inhibitor or the angiogenesis inhibitor is administered to the eye of the patient via an intravitreal injection. In some embodiments, the composition comprising the effective amount of the neovascularization inhibitor is administered at each of the multiple dosing sessions.

It is to be understood that one, some, or all of the properties of the various embodiments described herein may be combined to form other embodiments of the present invention. These and other aspects of the invention will become apparent to one of skill in the art.

### BRIEF DESCRIPTION OF THE DRAWINGS

FIG. 1A is a diagram of a general immunoconjugate dimer embodiment of provided herein.

FIG. 1B is a drawing of one embodiment of the immunoconjugate dimer of the present disclosure.

FIG. 2 is a graph of the rate of the intrinsic factor Xase complex (Fxase) hydrolysis (increase in absorbance at 405 nm-mOD/min) as a function of time.

FIG. 3 is a graph of thrombin generation by the known inhibitor of coagulation, active site inhibited FVIIa (FVIIai), as a function of time in normal pooled plasma.

FIG. 4 is a graph of thrombin generation by hI-con1, as a function of time in normal pooled plasma.

FIG. 5 is a graph of thrombin generation by human Factor VIIa and hI-con1 as a function of time in FVII-depleted plasma.

FIG. 6 is a graph of thrombin generation by hI-con1, as a function of time in rabbit plasma.

FIG. 7 is a graph of thrombin generation by hI-con1 or FVIIai as a function of time in centrifuged rabbit plasma.

FIG. 8 is a graph showing the percent CNV in the pig as a function of intravitreal dose of hI-con1. Intravitreal injections (100 μL/eye) of solutions of hI-con1 (0.25, 0.5, 1.0 and 2.0 mg/mL) were injected into both eyes of mini-pigs on Day 10; control animals received 100 μL of formulation buffer. On Day 14 the animals were sacrificed and the % CNV was determined.

FIG. 9 is a graph showing the percent CNV in the pig as a function of intravitreal dose of a 100 kDa fragment of hI-con1. Intravitreal injections (100 μL/eye) of solutions of hI-con1 (0.25, 0.5, 1.0 and 2.0 mg/mL) were injected into both eyes of mini-pigs on Day 10; control animals received 100 μL of formulation buffer. On Day 14 the animals were sacrificed and the % CNV was determined.

### DETAILED DESCRIPTION OF THE INVENTION

The abnormal growth of existing blood vessels and the creation of new blood vessels (referred to herein collectively as neovascularization), is observed in a variety of diseases, typically triggered by the release of specific growth factors for vascular endothelial cells. Neovascularization can enable solid tumor growth and metastasis, cause visual malfunction in ocular disorders, promote leukocyte extravasation in inflammatory disorders, and/or influence the outcome of cardiovascular diseases such as atherosclerosis.

Provided herein are immunoconjugate dimers and methods for use in treating a patient having a disease associated with pathological neovascularization including, but not limited to atherosclerosis, rheumatoid arthritis, ocular melanoma, solid tumor, primary or metastatic solid tumors (including but not limited to melanoma, renal, prostate, breast, ovarian, brain, neuroblastoma, head and neck, pancreatic, bladder, endometrial and lung cancer), diabetic macular edema (DME), macular edema following retinal vein occlusion (RVO), proliferative diabetic retinopathy, wet age-related macular degeneration (AMD), retinopathy of prematurity (ROP), and neovascular glaucoma. The immunoconjugate dimers of the invention target and bind to the Tissue Factor (TF) in diseased tissue, tumors, and the supporting stroma (vasculature, infiltrating mononuclear cells). As described herein, each monomer subunit of the immunoconjugate dimer comprises a targeting domain and an effector domain wherein the targeting domain and the effector domain are conjugated. The targeting domain of the immunoconjugate dimers comprise a mutated FVIIa protein. The effector domain of the immunoconjugate dimers comprise an Fc effector domain of an IgG1 immunoglobulin.

### Immunoconjugate Dimers

Provided herein are immunoconjugate dimers that target and bind to TF in diseased tissue, tumors, and the supporting stroma (vasculature, infiltrating mononuclear cells), and also bind Fc receptors.

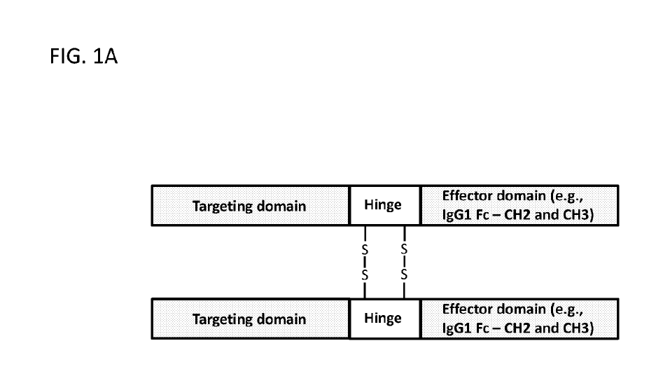

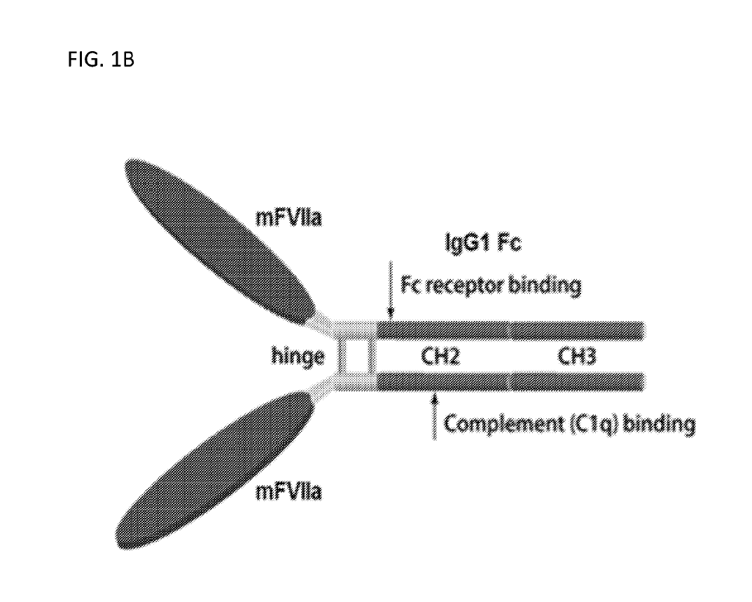

The immunoconjugates comprise a protein dimer having two monomer chains (interchangeably referred to herein as monomers or monomer subunits), wherein each monomer comprises a mutated Factor VIIa domain conjugated to an effector domain comprising the Fc region of an IgG1 immunoglobulin. FIGS. 1A and 1B provide generalized structures of the immunoconjugates provided herein (i.e., a protein dimer comprising two monomers each having a targeting domain conjugated to an effector domain).

The immunoconjugates of the invention are dimeric proteins. Each monomer of the dimer comprises a targeting domain and an effector domain wherein the targeting domain and the effector domain are conjugated. In one embodiment the effector and targeting domain are conjugated together by a hinge domain (interchangeably referred to herein as a hinge region). As presented herein, in some embodiments the effector domain is inclusive of a hinge region. In other embodiments, the effector domain does not include a hinge region. In some embodiments, the conjugation further comprises the inclusion of a linker. The targeting domain of the immunoconjugate dimers comprise a mutated FVIIa protein (tissue factor targeting domain). The effector domain of the immunoconjugate dimers comprise an Fc effector moiety of an IgG1 immunoglobulin. In one embodiment the targeting domain is a mutated human FVIIa protein and the effector domain is a human Fc effector moiety of an IgG1 immunoglobulin. In another embodiment the targeting domain is a mutated human FVIIa protein and the effector domain is a non-human Fc effector moiety of an IgG1 immunoglobulin. In one embodiment the targeting domain is a non-mutated human FVIIa protein and the effector domain is a human Fc effector moiety of an IgG1 immunoglobulin. In one embodiment the targeting domain is a non-mutated human FVIIa protein and the effector domain is a non-human (from the same species as the targeting domain) Fc effector moiety of an IgG1 immunoglobulin. In one embodiment the targeting domain is a non-mutated human FVIIa protein and the effector domain is a non-human (from a different species as that of the targeting domain) Fc effector moiety of an IgG1 immunoglobulin.

As provided herein, the immunoconjugate dimer binds to TF, but does not initiate/exhibits decreased initiation of the clotting cascade. The immunoconjugate dimer comprising the mutated FVIIa protein is designed such that FVIIa's normal role to initiate the clotting cascade does not occur or is reduced.

As provided throughout, in embodiments described herein, an immunoconjugate comprising a tissue factor targeting domain comprising a mutated Factor VIIa domain is provided. The targeting domain comprises a mutated Factor VIIa that has been mutated to inhibit (or reduce) initiation of the coagulation pathway without reducing binding affinity to tissue factor. In one embodiment, the mutation in human Factor VIIa is a single point mutation at residue 341. In a further embodiment, the mutation in human Factor VIIa is from Lys341 to Ala341. In other embodiments, where the mutant Factor VIIa is from a non-human species, it can comprise a mutation that corresponds to a mutation at residue 341 of the human Factor VIIa. Other mutations that inhibit the coagulation pathway are encompassed by the immunoconjugates provided herein. The mutated Factor VIIa domain (also referred to as the TF targeting domain), in the aspects provided herein, binds tissue factor with high affinity and specificity, but does not initiate coagulation, or minimizes coagulation normally associated with tissue factor binding.

The effector domain of the immunoconjugates provided herein comprise a Fc effector moiety of an IgG1 immunoglobulin. In one embodiment, the effector domain mediates both complement and natural killer (NK) cell cytotoxicity pathways. In one embodiment, cytotoxicity of immunologic cells such as NK cells and macrophages are activated by activating the Fc effector moiety when bound to Fc receptors present on cells of the immune system. The IgG1 Fc effector domain can trigger a cytolytic response against cells which bind the immunoconjugate, by the natural killer (NK) cell and complement pathways. In one embodiment, the IgG1 Fc effector domain comprises both the CH2 and CH3 regions of the IgG1 Fc region.

The reaction between FVIIa and TF is species-specific (Janson et al., 1984; Schreiber et al., 2005; Peterson et al., 2005): murine FVII appears to be active in many heterologous species including rabbits, pigs and humans, whereas human FVIIa is appreciably active in humans, non-human primates, dogs, rabbits, and pigs. Conversely, the human IgG Fc domain is active in both humans and mice. Accordingly, depending on the patient, the immunoconjugate is constructed using targeting and effector domains derived from the corresponding species, or from a species that is known to be active in the patient. For example, in the human treatment methods provided herein, the mutated tissue factor targeting domain can be derived from human Factor VIIa conjugated to an effector domain comprising the Fc region of a human IgG1 immunoglobulin.

Exemplary sequences for components of the immunoconjugate dimers described herein are provided in Table 1.

In one embodiment of the invention, a monomer of the immunoconjugate is or comprises a protein of SEQ ID NO: 2, 3, 11, 12, 13 or 14. In a further embodiment, a monomer of the immunoconjugate is or comprises a protein of SEQ ID NO: 2. In one embodiment, a monomer of the immunoconjugate is encoded by the sequence of SEQ ID NO: 1, 4 or 5.

In one embodiment, a monomer of the immunoconjugate is or comprises a protein of SEQ ID NO:2. In one embodiment, a monomer of the immunoconjugate of the invention consists of the sequence of SEQ ID NO:2.

In one embodiment, a monomer of the immunoconjugate is or comprises a protein of SEQ ID NO:3. In one embodiment, a monomer of the immunoconjugate of the invention consists of the sequence of SEQ ID NO:3.

In one embodiment, a monomer of the immunoconjugate is or comprises a protein of SEQ ID NO: 11. In one embodiment, a monomer of the immunoconjugate of the invention consists of the sequence of SEQ ID NO: 11.

In one embodiment, a monomer of the immunoconjugate is or comprises a protein of SEQ ID NO:12. In one embodiment, a monomer of the immunoconjugate of the invention consists of the sequence of SEQ ID NO: 12.

In one embodiment, a monomer of the immunoconjugate is or comprises a protein of SEQ ID NO: 13. In one embodiment, a monomer of the immunoconjugate of the invention consists of the sequence of SEQ ID NO:13.

In one embodiment, a monomer of the immunoconjugate is or comprises a protein of SEQ ID NO: 14. In one embodiment, a monomer of the immunoconjugate of the invention consists of the sequence of SEQ ID NO:14.

In one embodiment, the targeting domain of a monomer of the immunoconjugate is or comprises a protein of SEQ ID NO: 17. In one embodiment, the targeting domain of a monomer of the immunoconjugate consists of a protein of SEQ ID NO: 17.

In one embodiment, the targeting domain of a monomer of the immunoconjugate is or comprises a protein of SEQ ID NO: 18. In one embodiment, the targeting domain of a monomer of the immunoconjugate consists of a protein of SEQ ID NO: 18.

In one embodiment, the effector domain+hinge region of a monomer of the immunoconjugate comprises the sequence of SEQ ID NO: 19. In one embodiment, the effector domain+hinge region of a monomer of the immunoconjugate consists of the sequence of SEQ ID NO:19.

In one embodiment, the effector domain+hinge region of a monomer of the immunoconjugate comprises the sequence of SEQ ID NO:20. In one embodiment, the effector domain+hinge region of a monomer of the immunoconjugate consists of the sequence of SEQ ID NO:20.

In one embodiment, the effector domain+hinge region of a monomer of the immunoconjugate comprises the sequence of SEQ ID NO:21. In one embodiment, the effector domain+hinge region of a monomer of the immunoconjugate consists of the sequence of SEQ ID NO:21.

In one embodiment, the effector domain+hinge region of a monomer of the immunoconjugate comprises the sequence of SEQ ID NO:22. In one embodiment, the effector domain+hinge region of a monomer of the immunoconjugate consists of the sequence of SEQ ID NO:22.

In one embodiment, the effector domain+hinge region of a monomer of the immunoconjugate comprises the sequence of SEQ ID NO:23. In one embodiment, the effector domain+hinge region of a monomer of the immunoconjugate consists of the sequence of SEQ ID NO:23.

In one embodiment, the effector domain+hinge region of a monomer of the immunoconjugate comprises the sequence of SEQ ID NO:24. In one embodiment, the effector domain+hinge region of a monomer of the immunoconjugate consists of the sequence of SEQ ID NO:24.

In one embodiment a targeting domain comprising the sequence of SEQ ID NO: 17 and is conjugated to an effector domain (inclusive of a hinge region), wherein the effector domain (inclusive of a hinge region) comprises the sequence of SEQ ID NO: 19, SEQ ID NO:20, SEQ ID NO:21, SEQ ID NO:22, SEQ ID NO:23, or SEQ ID NO:24.

In one embodiment a targeting domain comprising the sequence of SEQ ID NO: 18 and is conjugated to an effector domain (inclusive of a hinge region), wherein the effector domain (inclusive of a hinge region) comprises the sequence of SEQ ID NO: 19, SEQ ID NO:20, SEQ ID NO:21, SEQ ID NO:22, SEQ ID NO:23, or SEQ ID NO:24.

TABLE 1
Exemplary Immunoconjugate Sequences

| SEQ ID | Description |
|---|---|
| SEQ ID NO: 1 | Homo sapiens Factor VII active site mutant immunoconjugate mRNA, |
| | complete coding sequence; NCBI Accession AF272774 (includes prop- |
| | peptide and signal sequence) |
| SEQ ID NO: 2 | Homo sapiens Factor VII active site mutant immunoconjugate amino acid |
| | sequence |
| SEQ ID NO: 3 | Homo sapiens Factor VII active site mutant immunoconjugate amino acid |
| | sequence, S344A and A341K (relative to SEQ ID NO: 2) |
| SEQ ID NO: 4 | Homo sapiens Factor VII active site mutant immunoconjugate coding |
| | sequence |
| SEQ ID NO: 5 | Homo sapiens Factor VII active site mutant immunoconjugate coding |
| | sequence |
| SEQ ID NO: 6 | Hinge region amino acid sequence |
| SEQ ID NO: 7 | Mutant hinge region amino acid sequence; N-terminal cysteine mutated to |
| | serine (relative to SEQ ID NO: 6) |
| SEQ ID NO: 8 | Mutant hinge region amino acid sequence; N-terminal lysine mutated to |
| | alanine (relative to SEQ ID NO: 7) |
| SEQ ID NO: 9 | Mutant hinge region amino acid sequence; deletion of residues 13-17 |
| | (relative to SEQ ID NO: 6) |
| SEQ ID NO: 10 | Mutant hinge region amino acid sequence; N-terminal serine mutated to |
| | glycine and deletion of residues 12-15 (relative to SEQ ID NO: 7) |
| SEQ ID NO: 11 | Homo sapiens Factor VII active site mutant immunoconjugate amino acid |
| | sequence comprising hinge region of SEQ ID NO: 7 |
| SEQ ID NO: 12 | Homo sapiens Factor VII active site mutant immunoconjugate amino acid |
| | sequence comprising hinge region of SEQ ID NO: 8 |
| SEQ ID NO: 13 | Homo sapiens Factor VII active site mutant immunoconjugate amino acid |
| | sequence comprising hinge region of SEQ ID NO: 9 |
| SEQ ID NO: 14 | Homo sapiens Factor VII active site mutant immunoconjugate amino acid |
| | sequence comprising hinge region of SEQ ID NO: 10 |
| SEQ ID NO: 15 | IgG Fc effector domain (comprising portion of a hinge) |
| SEQ ID NO: 16 | Human Factor VII active site mutant (targeting domain, comprising portion |
| | of a hinge) |
| SEQ ID NO: 17 | Homo sapiens Factor VII active site mutant amino acid sequence (targeting |
| | domain, no hinge sequence) |
| SEQ ID NO: 18 | Homo sapiens Factor VII active site mutant amino acid sequence, with |
| | mutations S344A and A341K relative to SEQ ID NO: 17 (targeting domain, |
| | no hinge sequence) |
| SEQ ID NO: 19 | Homo sapiens IgG Fc (effector domain comprising hinge sequence) |
| SEQ ID NO: 20 | Homo sapiens IgG Fc (effector domain comprising hinge sequence), Fc |
| | comprises ProtA mutation |
| SEQ ID NO: 21 | Homo sapiens IgG Fc (effector domain comprising mutated hinge region of |
| | SEQ ID NO: 7) |
| SEQ ID NO: 22 | Homo sapiens IgG Fc (effector domain comprising mutated hinge region of |
| | SEQ ID NO: 8) |
| SEQ ID NO: 23 | Homo sapiens IgG Fc (effector domain comprising mutated hinge region of |
| | SEQ ID NO: 9) |
| SEQ ID NO: 24 | Homo sapiens IgG Fc (effector domain comprising mutated hinge region of |
| | SEQ ID NO: 10) |

In one embodiment, the immunoconjugate comprises two protein chains, each comprising a targeting domain joined to an effector domain via a hinge region (or hinge domain). In a further embodiment, the hinge region is naturally occurring, and in one embodiment, is of human origin. In one embodiment, the hinge region of an IgG1 immunoglobulin, for example the hinge region of the human IgG1 immunoglobulin, is used to link the targeting domain to the effector domain. In one embodiment, the hinge region of IgG1 includes cysteine amino acids which form one or more disulfide bonds between the two monomer chains (e.g., as depicted in FIG. 1A). In one embodiment, the hinge region comprises the amino acid sequence of SEQ ID NO: 6, 7, 8, 9, or 10. In one embodiment, the effector domain comprises the sequence of SEQ ID NO: 19, 20, 21, 22, 23, or 24 (the effector domain inclusive of a hinge region sequence).

In one embodiment, the hinge region of the immunoconjugate of SEQ ID NO: 2 or 3 is altered to improve manufacturability of the immunoconjugate without affecting the binding properties to TF or Fc-receptors, all the while maintaining flexibility of the region. In one embodiment, the hinge regions of SEQ ID NO: 6, 7, 8, 9, or 10 confer improved manufacturability.

In some embodiments, a naturally occurring hinge region is modified. Without wishing to be bound by theory, it is thought that such a modification can aid in the yield of the immunoconjugate. In one embodiment, the hinge region of the immunoconjugate comprises SEQ ID NO:6. In one embodiment, the hinge region of the immunoconjugate comprises SEQ ID NO:7, which differs from SEQ ID NO:6 in that the n-terminal cysteine of SEQ ID NO:6 is mutated to a serine. In one embodiment, the hinge region of the immunoconjugate comprises SEQ ID NO:8, which differs from SEQ ID NO:7 in that the n-terminal lysine is mutated to alanine. In one embodiment, the hinge region of the immunoconjugate comprises SEQ ID NO:9, which differs from SEQ ID NO:6 in that residues 13-17 of SEQ ID NO:6 are deleted. In one embodiment, the hinge region of the immunoconjugate comprises SEQ ID NO: 10, which differs from SEQ ID NO:7 in that the n-terminal serine is mutated to a glycine and residues 12-15 of SEQ ID NO:7 are deleted.

In one embodiment, the immunoconjugate is a homodimer. However, in another embodiment, the immunoconjugate is a heterodimer, for example, an immunoconjugate comprising two monomers each having a targeting domain of a different amino acid sequence, but the same effector domains. The amino acid sequences of the two targeting domains, can differ by one amino acid, two or more amino acids, three or more amino acids or five or more amino acids. In one embodiment, each monomer subunit comprises an IgG1 hinge region that links the targeting region and effector region of the immunoconjugate, and the monomer subunits of the immunoconjugate heterodimer or the immunoconjugate homodimer are linked together via a disulfide bond between IgG1 hinge regions.

In one embodiment, the molecular weight of the immunoconjugate provided herein is from about 150 kDa to about 200 kDa. In another embodiment, the molecular weight of the immunoconjugate is about 157 kDa or is 157 kDa. For example, the immunoconjugate in one embodiment is the immunoconjugate having the amino acid sequence set forth in SEQ ID NO: 2, also referred to herein as “hI-con1.” In another embodiment, the immunoconjugate has the amino acid sequence set forth in SEQ ID NO: 3.

With respect to the sequences presented in Table 1, sequences with at least 80%, 85%, 90%, 95%, 96%, 97%, 98%, or even 99% percent identity to the sequences provided are contemplated.

In one embodiment, methods of producing the immunoconjugate include expression in BHK, HEK 293, CHO, and SP2/0 cells. Immunoconjugate monomers can be produced as fusion proteins or produced as chemical conjugates.

### Methods of the Invention

Provided herein are methods for using the immunoconjugate dimers of the invention, for treating a patient having a disease or disorder associated with neovascularization (e.g. tumor-associated neovascularization, such as cancer). The methods provided herein comprise administering to the patient a therapeutically effective amount of one or more immunoconjugate dimers provided herein for the treatment of a disease associated with pathological neovascularization including, but not limited to atherosclerosis, rheumatoid arthritis, ocular melanoma, solid tumor, primary or metastatic solid tumors (including but not limited to melanoma, renal, prostate, breast, ovarian, brain, neuroblastoma, head and neck, pancreatic, bladder, endometrial and lung cancer), diabetic macular edema (DME), macular edema following retinal vein occlusion (RVO), proliferative diabetic retinopathy, wet age-related macular degeneration (AMD), retinopathy of prematurity (ROP), and neovascular glaucoma. The immunoconjugates provided herein are amenable for use in any disease or disorder in which neovascularization is implicated. In one embodiment, methods for treating a patient for any cancer are provided. In one embodiment, methods for treating ocular melanoma are provided.

As used herein, the term “patient” includes both humans and other species, including other mammal species. The invention thus has both medical and veterinary applications. In veterinary compositions and treatments, immunoconjugates are constructed using targeting and effector domains derived from the corresponding species.

In one aspect, an immunoconjugate dimer provided herein is administered to the eye of a patient in need of treatment of ocular melanoma. In one embodiment, the treatment comprises multiple dosing sessions of the immunoconjugate dimer. In one embodiment, a monomer unit of the immunoconjugate dimer has the amino acid sequence of SEQ ID NO: 2, 3, 11, 12, 13, or 14. In a specific embodiment, a monomer unit of the immunoconjugate dimer has the amino acid sequence of SEQ ID NO: 2. In another specific embodiment, a monomer unit of the immunoconjugate dimer has the amino acid sequence of SEQ ID NO: 3. In another specific embodiment, a monomer unit of the immunoconjugate dimer has the amino acid sequence of SEQ ID NO: 11. In another specific embodiment, a monomer unit of the immunoconjugate dimer has the amino acid sequence of SEQ ID NO: 12. In another specific embodiment, a monomer unit of the immunoconjugate dimer has the amino acid sequence of SEQ ID NO: 13. In another specific embodiment, a monomer unit of the immunoconjugate dimer has the amino acid sequence of SEQ ID NO: 14. In another specific embodiment, a monomer unit of the immunoconjugate dimer comprises the targeting domain of SEQ ID NO: 17 or SEQ ID NO: 18 and comprises effector domain (inclusive of a hinge region) of SEQ ID NO: 19, SEQ ID NO:20, SEQ ID NO:21, SEQ ID NO:22, SEQ ID NO:23, or SEQ ID NO:24. In one aspect, the immunoconjugate dimer provided herein is administered to treat a metastasis of ocular melanoma. Such metastasis includes metastatic events that occur distal to the eye, i.e., liver, lung, bone, skin, brain, lymph nodes, and adrenal tissues.

In one aspect, an immunoconjugate dimer provided herein is administered to the eye of a patient in need of treatment of wet AMD. In one embodiment, the treatment comprises multiple dosing sessions of the immunoconjugate dimer. In one embodiment, a monomer unit of the immunoconjugate dimer has the amino acid sequence of SEQ ID NO: 2, 3, 11, 12, 13, or 14. In a specific embodiment, a monomer unit of the immunoconjugate dimer has the amino acid sequence of SEQ ID NO: 2. In another specific embodiment, a monomer unit of the immunoconjugate dimer has the amino acid sequence of SEQ ID NO: 3. In another specific embodiment, a monomer unit of the immunoconjugate dimer has the amino acid sequence of SEQ ID NO: 11. In another specific embodiment, a monomer unit of the immunoconjugate dimer has the amino acid sequence of SEQ ID NO: 12. In another specific embodiment, a monomer unit of the immunoconjugate dimer has the amino acid sequence of SEQ ID NO: 13. In another specific embodiment, a monomer unit of the immunoconjugate dimer has the amino acid sequence of SEQ ID NO: 14. In another specific embodiment, a monomer unit of the immunoconjugate dimer comprises the targeting domain of SEQ ID NO: 17 or SEQ ID NO: 18 and comprises effector domain (inclusive of a hinge region) of SEQ ID NO: 19, SEQ ID NO:20, SEQ ID NO:21, SEQ ID NO:22, SEQ ID NO:23, or SEQ ID NO:24.

In one embodiment, the method of treating wet AMD comprises preventing, inhibiting or reversing choroidal neovascularization in the eye of the patient in need of treatment. In a further embodiment, choroidal neovascularization is reversed by at least about 10%, at least about 20%, at least about 30% or at least about 40% after treatment, as compared to the choroidal neovascularization that was present in the afflicted eye of the patient prior to treatment.

Other ocular disorders associated with ocular neovascularization are treatable with the immunoconjugates and methods provided herein. The ocular neovascularization, in one embodiment, is choroidal neovascularization. In another embodiment the ocular neovascularization is retinal neovascularization. In yet another embodiment, the ocular neovascularization is corneal neovascularization. In yet another embodiment, the ocular neovascularization is an tumor-associated neovascularization of the eye. Accordingly, in one embodiment, an ocular disorder associated with choroidal, retinal or corneal neovascularization is treatable by one or more of the methods provided herein. In a further embodiment, the method comprises administering to the eye of a patient in need thereof, one of the immunoconjugate dimers described herein. In a further embodiment, the treatment comprises multiple dosing sessions of the immunoconjugate dimer. In one embodiment, a monomer unit of the immunoconjugate dimer has the amino acid sequence of SEQ ID NO: 2, 3, 11, 12, 13, or 14. In a specific embodiment, a monomer unit of the immunoconjugate dimer has the amino acid sequence of SEQ ID NO: 2. In another specific embodiment, a monomer unit of the immunoconjugate dimer has the amino acid sequence of SEQ ID NO: 3. In another specific embodiment, a monomer unit of the immunoconjugate dimer has the amino acid sequence of SEQ ID NO: 11. In another specific embodiment, a monomer unit of the immunoconjugate dimer has the amino acid sequence of SEQ ID NO: 12. In another specific embodiment, a monomer unit of the immunoconjugate dimer has the amino acid sequence of SEQ ID NO: 13. In another specific embodiment, a monomer unit of the immunoconjugate dimer has the amino acid sequence of SEQ ID NO: 14. In another specific embodiment, a monomer unit of the immunoconjugate dimer comprises the targeting domain of SEQ ID NO: 17 or SEQ ID NO: 18 and comprises effector domain (inclusive of a hinge region) of SEQ ID NO: 19, SEQ ID NO:20, SEQ ID NO:21, SEQ ID NO:22, SEQ ID NO:23, or SEQ ID NO:24.

For example, in one embodiment, a patient in need of treatment of proliferative diabetic retinopathy, wet age-related macular degeneration (AMD), retinopathy of prematurity (ROP), or neovascular glaucoma is treated with one of the immunoconjugates provided herein, for example, via intravitreal injection, suprachoroidal injection or topical administration (e.g., via eyedrops) of the immunoconjugate into the affected eye. Treatment in one embodiment occurs over multiple dosing sessions. With respect to the aforementioned disorders, ocular neovascularization is said to be “associated with” or “secondary to” the respective disorder.

In one embodiment, a patient in need of treatment of macular edema following retinal vein occlusion (RVO) is treated by one of the immunoconjugate dimers provided herein. In one embodiment, the treatment comprises multiple dosing sessions of the immunoconjugate dimer. In one embodiment, a monomer unit of the immunoconjugate dimer has the amino acid sequence of SEQ ID NO: 2, 3, 11, 12, 13, or 14. In a specific embodiment, a monomer unit of the immunoconjugate dimer has the amino acid sequence of SEQ ID NO: 2. In another specific embodiment, a monomer unit of the immunoconjugate dimer has the amino acid sequence of SEQ ID NO: 3. In another specific embodiment, a monomer unit of the immunoconjugate dimer has the amino acid sequence of SEQ ID NO: 11. In another specific embodiment, a monomer unit of the immunoconjugate dimer has the amino acid sequence of SEQ ID NO: 12. In another specific embodiment, a monomer unit of the immunoconjugate dimer has the amino acid sequence of SEQ ID NO: 13. In another specific embodiment, a monomer unit of the immunoconjugate dimer has the amino acid sequence of SEQ ID NO: 14. In another specific embodiment, a monomer unit of the immunoconjugate dimer comprises the targeting domain of SEQ ID NO: 17 or SEQ ID NO: 18 and comprises effector domain (inclusive of a hinge region) of SEQ ID NO: 19. SEQ ID NO:20, SEQ ID NO:21, SEQ ID NO:22, SEQ ID NO:23, or SEQ ID NO:24. In one embodiment, the immunoconjugate dimer is administered to the patient over multiple dosing sessions, for example, via intravitreal administration at each dosing session.

In another embodiment, a patient in need of treatment of diabetic macular edema (DME) is treated by one of the immunoconjugate dimers provided herein. In one embodiment, the treatment comprises multiple dosing sessions of the immunoconjugate dimer. In one embodiment, a monomer unit of the immunoconjugate dimer has the amino acid sequence of SEQ ID NO: 2, 3, 11, 12, 13, or 14. In a specific embodiment, a monomer unit of the immunoconjugate dimer has the amino acid sequence of SEQ ID NO: 2. In another specific embodiment, a monomer unit of the immunoconjugate dimer has the amino acid sequence of SEQ ID NO: 3. In another specific embodiment, a monomer unit of the immunoconjugate dimer has the amino acid sequence of SEQ ID NO: 11. In another specific embodiment, a monomer unit of the immunoconjugate dimer has the amino acid sequence of SEQ ID NO: 12. In another specific embodiment, a monomer unit of the immunoconjugate dimer has the amino acid sequence of SEQ ID NO: 13. In another specific embodiment, a monomer unit of the immunoconjugate dimer has the amino acid sequence of SEQ ID NO: 14. In another specific embodiment, a monomer unit of the immunoconjugate dimer comprises the targeting domain of SEQ ID NO: 17 or SEQ ID NO: 18 and comprises effector domain (inclusive of a hinge region) of SEQ ID NO: 19, SEQ ID NO:20, SEQ ID NO:21, SEQ ID NO:22, SEQ ID NO:23, or SEQ ID NO:24. In one embodiment, the immunoconjugate dimer is administered to the patient over multiple dosing sessions, for example, via intravitreal administration at each dosing session. In even a further embodiment, the immunoconjugate dimer is administered intravitreally at each dosing session.

In yet another embodiment, diabetic retinopathy is treated via one of the immunoconjugates provided herein, in a patient in need thereof, for example, a patient with DME. In one embodiment, the treatment comprises multiple dosing sessions of the immunoconjugate dimer. In one embodiment, a monomer unit of the immunoconjugate dimer has the amino acid sequence of SEQ ID NO: 2, 3, 11, 12, 13, or 14. In a specific embodiment, a monomer unit of the immunoconjugate dimer has the amino acid sequence of SEQ ID NO: 2. In another specific embodiment, a monomer unit of the immunoconjugate dimer has the amino acid sequence of SEQ ID NO: 3. In another specific embodiment, a monomer unit of the immunoconjugate dimer has the amino acid sequence of SEQ ID NO: 11. In another specific embodiment, a monomer unit of the immunoconjugate dimer has the amino acid sequence of SEQ ID NO: 12. In another specific embodiment, a monomer unit of the immunoconjugate dimer has the amino acid sequence of SEQ ID NO: 13. In another specific embodiment, a monomer unit of the immunoconjugate dimer has the amino acid sequence of SEQ ID NO: 14. In another specific embodiment, a monomer unit of the immunoconjugate dimer comprises the targeting domain of SEQ ID NO: 17 or SEQ ID NO: 18 and comprises effector domain (inclusive of a hinge region) of SEQ ID NO: 19, SEQ ID NO:20, SEQ ID NO:21, SEQ ID NO:22, SEQ ID NO:23, or SEQ ID NO:24. In one embodiment, the immunoconjugate dimer is administered to the patient over multiple dosing sessions, for example, via intravitreal administration at each dosing session. In even a further embodiment, the immunoconjugate dimer is administered to the patient over multiple dosing sessions, for example, via intravitreal administration at each dosing session.

In one embodiment of the invention, one or more of the immunoconjugates provided herein is used in a method to treat a disease or disorder associated with tumor neovascularization in a patient in need thereof, for example, a cancer patient. In one embodiment, the method comprises administering to the patient, for example via intratumoral or intravenous injection, a composition comprising a therapeutically effective amount of an immunoconjugate dimer of the invention. In one embodiment, the treatment comprises multiple dosing sessions of the immunoconjugate dimer. In one embodiment, a monomer unit of the immunoconjugate dimer has the amino acid sequence of SEQ ID NO: 2, 3, 11, 12, 13, or 14. In a specific embodiment, a monomer unit of the immunoconjugate dimer has the amino acid sequence of SEQ ID NO: 2. In another specific embodiment, a monomer unit of the immunoconjugate dimer has the amino acid sequence of SEQ ID NO: 3. In another specific embodiment, a monomer unit of the immunoconjugate dimer has the amino acid sequence of SEQ ID NO: 11. In another specific embodiment, a monomer unit of the immunoconjugate dimer has the amino acid sequence of SEQ ID NO: 12. In another specific embodiment, a monomer unit of the immunoconjugate dimer has the amino acid sequence of SEQ ID NO: 13. In another specific embodiment, a monomer unit of the immunoconjugate dimer has the amino acid sequence of SEQ ID NO: 14. In another specific embodiment, a monomer unit of the immunoconjugate dimer comprises the targeting domain of SEQ ID NO: 17 or SEQ ID NO: 18 and comprises effector domain (inclusive of a hinge region) of SEQ ID NO: 19, SEQ ID NO:20, SEQ ID NO:21, SEQ ID NO:22, SEQ ID NO:23, or SEQ ID NO:24.

In cancer treatments, the immunoconjugate dimer is used for treating a variety of cancers, particularly primary or metastatic solid tumors, including but not limited to melanoma, renal, prostate, breast, ovarian, brain, neuroblastoma, head and neck, pancreatic, bladder, endometrial and lung cancer. In one embodiment, the cancer is a gynecological cancer. In a further embodiment, the gynecological cancer is serous, clear cell, endometriod or undifferentiated ovarian cancer. The immunoconjugate dimer in one embodiment is employed to target the tumor vasculature, particularly vascular endothelial cells, and/or tumor cells. Without wishing to be bound by theory, targeting the tumor vasculature can offer several advantages for cancer immunotherapy with one or more of the immunoconjugate dimers described herein, as follows. (i) some of the vascular targets including tissue factor should be the same for all tumors; (ii) immunoconjugates targeted to the vasculature do not have to infiltrate a tumor mass in order to reach their targets; (iii) targeting the tumor vasculature should generate an amplified therapeutic response, because each blood vessel nourishes numerous tumor cells whose viability is dependent on the functional integrity of the vessel; and (iv) the vasculature is unlikely to develop resistance to an immunoconjugate, because that would require modification of the entire endothelium layer lining a vessel. Unlike previously described antiangiogenic methods that inhibit new vascular growth, immunoconjugate dimers provided herein elicit a cytolytic response to the neovasculature.

In another embodiment, one or more of the immunoconjugates described herein is used in a method for treating atherosclerosis or rheumatoid arthritis. In one embodiment, the treatment comprises multiple dosing sessions of the immunoconjugate dimer. I In one embodiment, a monomer unit of the immunoconjugate dimer has the amino acid sequence of SEQ ID NO: 2, 3, 11, 12, 13, or 14. In a specific embodiment, a monomer unit of the immunoconjugate dimer has the amino acid sequence of SEQ ID NO: 2. In another specific embodiment, a monomer unit of the immunoconjugate dimer has the amino acid sequence of SEQ ID NO: 3. In another specific embodiment, a monomer unit of the immunoconjugate dimer has the amino acid sequence of SEQ ID NO: 11. In another specific embodiment, a monomer unit of the immunoconjugate dimer has the amino acid sequence of SEQ ID NO: 12. In another specific embodiment, a monomer unit of the immunoconjugate dimer has the amino acid sequence of SEQ ID NO: 13. In another specific embodiment, a monomer unit of the immunoconjugate dimer has the amino acid sequence of SEQ ID NO: 14. In another specific embodiment, a monomer unit of the immunoconjugate dimer comprises the targeting domain of SEQ ID NO: 17 or SEQ ID NO: 18 and comprises effector domain (inclusive of a hinge region) of SEQ ID NO:19, SEQ ID NO:20. SEQ ID NO:21, SEQ ID NO:22, SEQ ID NO:23, or SEQ ID NO:24.

In one embodiment of a method for treating an ocular disorder such as ocular melanoma with an immunoconjugate dimer, for example, a method for treating wet AMD, diabetic retinopathy, diabetic macular edema, or choroidal neovascularization secondary to an ocular disorder such as wet AMD, the patient subjected to the treatment method substantially maintains his or her vision subsequent to the treatment (e.g., the single dosing session or multiple dosing sessions), as measured by losing fewer than 15 letters in a best-corrected visual acuity (BCVA) measurement, compared to the patient's BCVA measurement prior to undergoing treatment. In a further embodiment, the patient loses fewer than 10 letters, fewer than 8 letters, fewer than 6 letters or fewer than 5 letters in a BCVA measurement, compared to the patient's BCVA measurement prior to undergoing treatment.

In another embodiment of a method for treating an ocular disorder with an immunoconjugate dimer, for example, a method for treating ocular melanoma, wet AMD, diabetic retinopathy, diabetic macular edema, tumor-associated neovascularization, or choroidal neovascularization secondary to an ocular disorder such as wet AMD, the patient subjected to the treatment method substantially maintains his or her vision subsequent to the treatment (e.g., the single dosing session or multiple dosing sessions), as measured by gaining 15 or more letters in a best-corrected visual acuity (BCVA) measurement, compared to the patient's BCVA prior to the multiple dosing sessions. In a further embodiment, the patient gains about 15 letters or more, about 20 letters or more, about 25 letters or more in a BCVA measurement, compared to the patient's BCVA measurement prior to undergoing treatment. In even a further embodiment, the patient gains from about 15 to about 30 letters, from about 15 letters to about 25 letters or from about 15 letters to about 20 letters in a BCVA measurement, compared to the patient's BCVA measurement prior to undergoing treatment.

In one embodiment of a method for treating an ocular disorder in the eye of a patient in need thereof with an immunoconjugate dimer, for example, a method for treating wet AMD, diabetic retinopathy, diabetic macular edema, or choroidal neovascularization secondary to an ocular disorder such as wet AMD provided herein, the ocular neovascularization area, e.g., the choroidal neovascularization area of the eye of the patient is reduced in the eye of the patient, as compared to the ocular neovascularization area (e.g., CNV area) prior to treatment. As provided herein, treatment can include one dosing session or multiple dosing sessions, and reduction in ocular neovascularization area (e.g., CNV area), in one embodiment, is assessed after individual dosing sessions, or multiple dosing sessions. In a further embodiment, the ocular neovascularization area (e.g., CNV area) is reduced by at least about 5%, or at least about 10%, or at least about 15%, or at least about 20%, or at least about 25%, or at least about 30%, or at least about 35%, or at least about 40%, or at least about 45%, or at least about 50%, as measured by fluorescein angiography.

In one embodiment of a method for treating an ocular disorder in the eye of a patient in need thereof with an immunoconjugate dimer, for example, a method for treating wet AMD, diabetic retinopathy, diabetic macular edema, or choroidal neovascularization secondary to an ocular disorder such as wet AMD provided herein, the retinal thickness of the treated eye is reduced in the eye of the patient, as compared to the retinal thickness prior to treatment, as measured by optical coherence tomography (OCT). As provided herein, treatment can include one dosing session or multiple dosing sessions, and reduction in retinal thickness, in one embodiment, is assessed after individual dosing sessions, or multiple dosing sessions. In a further embodiment, the retinal thickness is reduced by at least about 5%, or at least about 10%, or at least about 15%, or at least about 20%, or at least about 25%, or at least about 30%, or at least about 35%, or at least about 40%, or at least about 45%, or at least about 50%, as measured by OCT. In a further embodiment, the decreased retinal thickness is decreased central retinal subfield thickness (CST), decreased center point thickness (CPT), or decreased central foveal thickness (CFT).

In one embodiment of a method of the present disclosure, the patient exhibits a decrease in the size, volume, and/or thickness of the ocular melanoma subsequent to the one or more dosing sessions, as compared to the size, volume, and/or thickness of the ocular melanoma prior to the one or more dosing sessions. In a further embodiment, the decrease in size, volume, and/or thickness of the ocular melanoma is measured by ultrasound, high-resolution ultrasound biomicroscopy, magnetic resonance imaging, and/or computed axial tomography.

In one embodiment of an ocular melanoma treatment method provided herein, the patient exhibits a decrease in swelling and/or fluid accumulation beneath the retina or choroid subsequent to the one or more dosing sessions, as compared to the swelling and/or fluid accumulation beneath the retina or choroid prior to the one or more dosing session. In a further embodiment, the decrease in swelling and/or fluid accumulation is measured by ocular coherence tomography.

In one embodiment of a method of the present disclosure, the patient exhibits a decrease in in leakage or blockage of blood vessels in the eye subsequent to the one or more dosing sessions, as compared to the leakage or blockage of blood vessels in the eye prior to the one or more dosing sessions. In a further embodiment, the decrease is measured by fluorescein angiography or indocyanine green angiography.

In one embodiment of a method of the present disclosure, the patient exhibits a decrease in the size and/or number of iris spots subsequent to the one or more dosing sessions, as compared to the size and/or number of iris spots prior to the one or more dosing sessions. In a further embodiment, the decrease is measured by gonioscope, slit-lamp biomicroscope, and/or ophthalmoscope.

The immunoconjugates provided herein are amenable for use in any disease or disorder in which pathological neovascularization is implicated. For example, in one aspect, an immunoconjugate dimer provided herein is administered to the eye of a patient in need of treatment of ocular melanoma. In one embodiment, the treatment comprises multiple dosing sessions of the immunoconjugate dimer. As provided throughout, the immunoconjugate dimer comprises monomer subunits that each include a mutated human Factor VIIa (FVIIa) protein conjugated to the human immunoglobulin G1 (IgG1) Fc domain. In one embodiment, a monomer unit of the immunoconjugate dimer has the amino acid sequence of SEQ ID NO: 2, 3, 11, 12, 13, or 14. In a specific embodiment, a monomer unit of the immunoconjugate dimer has the amino acid sequence of SEQ ID NO: 2. In another specific embodiment, a monomer unit of the immunoconjugate dimer has the amino acid sequence of SEQ ID NO: 3. In another specific embodiment, a monomer unit of the immunoconjugate dimer has the amino acid sequence of SEQ ID NO: 11. In another specific embodiment, a monomer unit of the immunoconjugate dimer has the amino acid sequence of SEQ ID NO: 12. In another specific embodiment, a monomer unit of the immunoconjugate dimer has the amino acid sequence of SEQ ID NO: 13. In another specific embodiment, a monomer unit of the immunoconjugate dimer has the amino acid sequence of SEQ ID NO: 14. In another specific embodiment, a monomer unit of the immunoconjugate dimer comprises the targeting domain of SEQ ID NO: 17 or SEQ ID NO: 18 and comprises effector domain (inclusive of a hinge region) of SEQ ID NO: 19, SEQ ID NO:20, SEQ ID NO:21, SEQ ID NO:22, SEQ ID NO:23, or SEQ ID NO:24.

In one embodiment, the method of treating ocular melanoma comprises preventing, inhibiting or reversing tumor-associated neovascularization in the eye of the patient in need of treatment. In a further embodiment, neovascularization is reversed by at least about 10%, at least about 20%, at least about 30% or at least about 40% after treatment as compared to the choroidal neovascularization that was present in the afflicted eye of the patient prior to treatment.

In one embodiment of a method for treating an ocular melanoma with an immunoconjugate dimer, the patient subjected to the treatment method substantially maintains his or her vision subsequent to the treatment (e.g., the single dosing session or multiple dosing sessions), as measured by losing fewer than 15 letters in a best-corrected visual acuity (BCVA) measurement, compared to the patient's BCVA measurement prior to undergoing treatment. In a further embodiment, the patient loses fewer than 10 letters, fewer than 8 letters, fewer than 6 letters or fewer than 5 letters in a BCVA measurement, compared to the patient's BCVA measurement prior to undergoing treatment.

In another embodiment of a method for treating an ocular melanoma with an immunoconjugate dimer, the patient subjected to the treatment method substantially maintains his or her vision subsequent to the treatment (e.g., the single dosing session or multiple dosing sessions), as measured by gaining 15 or more letters in a best-corrected visual acuity (BCVA) measurement, compared to the patient's BCVA prior to the multiple dosing sessions. In a further embodiment, the patient gains about 15 letters or more, about 20 letters or more, about 25 letters or more in a BCVA measurement, compared to the patient's BCVA measurement prior to undergoing treatment. In even a further embodiment, the patient gains from about 15 to about 30 letters, from about 15 letters to about 25 letters or from about 15 letters to about 20 letters in a BCVA measurement, compared to the patient's BCVA measurement prior to undergoing treatment.

In one embodiment of a method for treating an ocular melanoma in the eye of a patient in need thereof with an immunoconjugate dimer provided herein, the ocular neovascularization area of the eye of the patient is reduced in the eye of the patient, as compared to the ocular neovascularization area prior to treatment. As provided herein, treatment can include one dosing session or multiple dosing sessions, and reduction in ocular neovascularization area (e.g., CNV area), in one embodiment, is assessed after individual dosing sessions, or multiple dosing sessions. In a further embodiment, the ocular neovascularization area (e.g., CNV area) is reduced by at least about 5%, or at least about 10%, or at least about 15%, or at least about 20%, or at least about 25%, or at least about 30%, or at least about 35%, or at least about 40%, or at least about 45%, or at least about 50%, as measured by fluorescein angiography.

In one embodiment of a method for treating an ocular melanoma in the eye of a patient in need thereof with an immunoconjugate dimer provided herein, the retinal thickness of the treated eye is reduced in the eye of the patient, as compared to the retinal thickness prior to treatment, as measured by optical coherence tomography (OCT). As provided herein, treatment can include one dosing session or multiple dosing sessions, and reduction in retinal thickness, in one embodiment, is assessed after individual dosing sessions, or multiple dosing sessions. In a further embodiment, the retinal thickness is reduced by at least about 5%, or at least about 10%, or at least about 15%, or at least about 20%, or at least about 25%, or at least about 30%, or at least about 35%, or at least about 40%, or at least about 45%, or at least about 50%, as measured by OCT. In a further embodiment, the decreased retinal thickness is decreased central retinal subfield thickness (CST), decreased center point thickness (CPT), or decreased central foveal thickness (CFT).

In one embodiment of a method of the present disclosure, the patient exhibits a decrease in the size, volume, and/or thickness of the ocular melanoma subsequent to the one or more dosing sessions, as compared to the size, volume, and/or thickness of the ocular melanoma prior to the one or more dosing sessions. In a further embodiment, the decrease in size, volume, and/or thickness of the ocular melanoma is measured by ultrasound, high-resolution ultrasound biomicroscopy, magnetic resonance imaging, and/or computed axial tomography.

In one embodiment of an ocular melanoma treatment method provided herein, the patient exhibits a decrease in swelling and/or fluid accumulation beneath the retina or choroid subsequent to the one or more dosing sessions, as compared to the swelling and/or fluid accumulation beneath the retina or choroid prior to the one or more dosing session. In a further embodiment, the decrease in swelling and/or fluid accumulation is measured by ocular coherence tomography.

In one embodiment of a method of the present disclosure, the patient exhibits a decrease in in leakage or blockage of blood vessels in the eye subsequent to the one or more dosing sessions, as compared to the leakage or blockage of blood vessels in the eye prior to the one or more dosing sessions. In a further embodiment, the decrease is measured by fluorescein angiography or indocyanine green angiography.

In one embodiment of a method of the present disclosure, the patient exhibits a decrease in the size and/or number of iris spots subsequent to the one or more dosing sessions, as compared to the size and/or number of iris spots prior to the one or more dosing sessions. In a further embodiment, the decrease is measured by gonioscope, slit-lamp biomicroscope, and/or ophthalmoscope.

### Administration and Dosing

Provided herein are administration and dosing embodiments for the immunoconjugate dimer (or a composition comprising the immunoconjugate dimer).

In one embodiment, the immunoconjugate dimer is administered as a solution or a suspension. The immunoconjugate dimer, in one embodiment, comprises arginine or protein A.

In a further embodiment, the immunoconjugate dimer comprises arginine. In even a further embodiment, the arginine is present in the composition at from about 20 mM to about 40 mM, e.g., at 25 mM. Other components of the composition, in one embodiment, include HEPES, sodium chloride, polysorbate-80, calcium chloride, or a combination thereof.

In one embodiment, the immunoconjugate dimer is administered in a dose of between 1 μg and 1500 μg, In one embodiment, the immunoconjugate dimer is administered in a dose of between 10 μg and 600 μg, 10 μg and 500 μg, 10 μg and 400 μg, 10 μg and 300 μg, 10 μg and 200 μg, 10 μg and 100 μg, 10 μg and 50 μg, 50 μg and 600 μg, 50 μg and 500 μg, 50 μg and 400 μg, 50 μg and 300 μg, 50 μg and 200 μg, 50 μg and 100 μg, 100 μg and 600 μg, 100 μg and 500 μg, 100 μg and 400 μg, 100 μg and 300 μg, 100 μg and 200 μg, 200 μg and 600 μg, 200 μg and 500 μg, 200 μg and 400 μg, 200 μg and 300 μg, 300 μg and 500 μg, 300 μg and 400 μg, or 400 μg and 500 μg. In one embodiment, the immunoconjugate dimer is administered at single dose of 300 μg. In one embodiment, the immunoconjugate dimer is administered with multiples doses of 300 μg each. In one embodiment, the immunoconjugate dimer is administered at single dose of 600 μg. In one embodiment, the immunoconjugate dimer is administered with multiples doses of 600 μg each.

In one embodiment, the immunoconjugate dimer is administered in a dose consisting of about 1 μg, 10 μg, about 20 μg, about 30 μg, about 40 μg, about 50 μg, about 60 μg, about 70 μg, about 80 μg, about 90 μg, about 100 μg, about 125 μg, about 150 μg, about 175 μg, about 200 μg, about 225 μg, about 250 μg, about 275 μg, about 300 μg, about 325 μg, about 350 μg, about 375 μg, about 400 μg, about 425 μg, about 450 μg, about 475 μg, about 500 μg, about 525 μg, about 550 μg, about 575 μg, about 600 μg, about 625 μg, about 650 μg, about 675 μg, about 700 μg, about 725 μg, about 750 μg, about 775 μg, about 800 μg, about 825 μg, about 850 μg, 875 μg, about 900 μg, about 925 μg, about 950 μg, about 975 μg, about 1000 μg, about 1100 μg, about 1200 μg, about 1300 μg, about 1400 μg, or about 1500 μg.

In some embodiments, a single dose of the immunoconjugate dimer is administered. In some embodiments, two or more doses of the immunoconjugate dimer is administered. In an exemplary embodiment, two doses of 300 μg each are administered, spaced by an interval of 1 week (7 days). In an exemplary embodiment, two doses of 600 μg each are administered, spaced by an interval of 1 week (7 days).

In one embodiment, the immunoconjugate dimer is administered in a solute volume of between 10 μL and 200 μL, 10 μL and 180 μL, 10 μL and 160 μL, 10 μL and 140 μL, 10 μL and 120 μL, 10 μL and 100 μL, 10 μL and 80 μL, 10 μL and 60 μL, 10 μL and 40 μL, 10 μL and 20 μL, 10 μL and 15 μL, 20 μL and 200 μL, 20 μL and 180 μL, 20 μL and 160 μL, 20 μL and 140 μL, 20 μL and 120 μL, 20 μL and 100 μL, 20 μL and 80 μL, 20 μL and 60 μL, 20 μL and 40 μL, 40 μL and 200 μL, 40 μL and 180 μL, 40 μL and 160 μL, 40 μL and 140 μL, 40 μL and 120 μL, 40 μL and 100 μL, 40 μL and 80 μL, 40 μL and 60 μL, 60 μL and 200 μL, 60 μL and 180 μL, 60 μL and 160 μL, 60 μL and 140 μL, 60 μL and 120 μL, 60 μL and 100 μL, 60 μL and 80 μL, 80 μL and 200 μL, 80 μL and 180 μL, 80 μL and 160 μL, 80 μL and 140 μL, 80 μL and 120 μL, 80 μL and 100 μL, 100 μL and 200 μL, 100 μL and 180 μL, 100 μL and 160 μL, 100 μL and 140 μL, 100 μL and 120 μL, 120 μL and 200 μL, 120 μL and 180 μL, 120 μL and 160 μL, 120 μL and 140 μL, 140 μL and 200 μL, 140 μL and 180 μL, 140 μL and 160 μL, 160 μL and 200 μL, 160 μL and 180 μL, or 180 μL and 200 μL.

In one embodiment, the immunoconjugate dimer is administered in a solute volume consisting of about 10 μL, about 15 μL, about 20 μL, about 25 μL, about 30 μL, about 35 μL, about 40 μL, about 45 μL, about 50 μL, about 55 μL, about 60 μL, about 65 μL, about 70 μL, about 75 μL, about 80 μL, about 85 μL, about 90 μL, about 95 μL, or about 100 μL.

Exemplary compositions of the present invention are provided in Table 2 and 3 below.

TABLE 2
Exemplary immunoconjugate dimer of the invention

| Component | Concentration |
|---|---|
| Immunoconjugate dimer | 3 mg/mL in 15 mM HEPES |
| NaCl | 150 mM |
| Arginine | 25 mM, pH 7.4 |
| Polysorbate-80 | 0.01% |
| CaCl₂ | 5 mM |

TABLE 3
Exemplary immunoconjugate dimer of the invention

| Component | Concentration |
|---|---|
| Immunoconjugate dimer of SEQ ID | 3 mg/mL in 15 mM HEPES |
| NO: 2, 3, 4, 5, 11, 12, 13 or 14 | |
| NaCl | 150 mM |
| Arginine | 25 mM, pH 7.4 |
| Polysorbate-80 | 0.01% |
| CaCl₂ | 5 mM |

Administration methods encompassed by the methods provided herein include intravitreal injection, suprachoroidal injection, topical administration (e.g., eye drops), intravenous and intratumoral administration. In another embodiment, administration is via intravenous, intramuscular, intratumoral, subcutaneous, intrasynovial, intraocular, intraplaque, or intradermal injection of the immunoconjugate or of a replication-deficient adenoviral vector, or other viral vectors carrying a cDNA encoding a secreted form of the immunoconjugate. In one embodiment, the patient in need of treatment is administered one or more immunoconjugate dimers via intravitreal, intravenous or intratumoral injection, or injection at other sites, of one or more immunoconjugate proteins. Alternatively, in one embodiment, a patient in need of treatment is administered one or more immunoconjugate dimers via intravenous or intratumoral injection, or injection at other sites, of one or more expression vectors carrying a cDNA encoding a secreted form of one or more of the immunoconjugate dimers provided herein. In some embodiments, the patient is treated by intravenous or intratumoral injection of an effective amount of one or more replication-deficient adenoviral vectors, or one or more adeno-associated vectors carrying cDNA encoding a secreted form of one or more types of immunoconjugate proteins.

As described herein, administration may be local or systemic, depending upon the type of pathological condition involved in the therapy.

In one embodiment, a method of intravitreal injection is employed. In a further embodiment, aseptic technique is employed when preparing the immunoconjugate dimer for injection, for example, via the use of sterile gloves, a sterile drape and a sterile eyelid speculum (or equivalent). In one embodiment, the patient is subjected to anesthesia and a broad-spectrum microbicide prior to the injection.

In one embodiment, intravitreal injection of one or more of the immunoconjugate dimers provided herein, for example the immunoconjugate dimer of SEQ ID NO: 2, 3, 11, 12, 13 or 14 is prepared by withdrawing the vial contents of the immunoconjugate dimer composition solution through a 5-micron, 19-gauge filter needle attached to a 1-cc tuberculin syringe. The filter needle in a further embodiment, is then discarded and replaced with a sterile 30-gauge×½V-inch needle for the intravitreal injection. The contents of the vial are expelled until the plunger tip is aligned with the line on the syringe that marks the appropriate dose for delivery.

In one method of ocular injection, e.g., intravitreal or suprachoroidal injection, prior to and/or after the injection, the patient is monitored for elevation in intraocular pressure (IOP). For example, in one embodiment, prior to and/or after the ocular injection, the patient is monitored for elevation in IOP using tonometry. In another embodiment, the patient is monitored for increases in IOP via a check for perfusion of the optic nerve head immediately after the injection. In one embodiment, prior to ocular injection of one of the immunoconjugate dimers provided herein, for example about 20 minutes, about 30 minutes, about 40 minutes, about 50 minutes or about 1 hour prior to the ocular injection, the patient is monitored for elevation in IOP. In another embodiment, after ocular injection of one of the immunoconjugate dimers provided herein, for example, about 10 minutes, about 20 minutes, about 30 minutes, about 40 minutes, about 50 minutes or about 1 hour after the intraocular injection, the patient is monitored for elevation in IOP. In one embodiment, the patient's IOP is substantially the same prior to intraocular injection of an immunoconjugate dimer, as compared to after intraocular injection of the immunoconjugate dimer. In one embodiment, the patient's IOP varies by no more than 10%, no more than 20% or no more than 30% after intraocular injection, as compared to prior to intraocular injection (e.g., intravitreal injection).

The treatment methods provided herein in one embodiment, comprise a single administration of one of the immunoconjugate dimers provided herein (e.g., an immunoconjugate of SEQ ID NO: 2, 3, 11, 12, 13 or 14). However, in another embodiment, the treatment methods provided herein comprise multiple dosing sessions. In a further embodiment, the multiple dosing sessions are multiple intraocular injections of one of the immunoconjugate dimers described herein. The multiple dosing sessions, in one embodiment comprise two or more, three or more, four or more or five or more dosing sessions. In a further embodiment, each dosing session comprises intraocular injection of one of the immunoconjugates described herein, or intratumoral injection of one of the immunoconjugates described herein (i.e., either as the expressed protein or via a vector encoding the soluble immunoconjugate).

In one embodiment, from about 2 to about 24 dosing sessions are employed, for example, from about 2 to about 24 intraocular dosing sessions (e.g., intravitreal or suprachoroidal injection). In a further embodiment, from about 3 to about 30, or from about 5 to about 30, or from about 7 to about 30, or from about 9 to about 30, or from about 10 to about 30, or from about 12 to about 30 or from about 12 to about 24 dosing sessions are employed.

In one embodiment, where multiple dosing sessions are employed, the dosing sessions are spaced apart by from about 0.5 days, 1 day, 3 days, 4 days, 5 days, 6 days, 7 days, 8 days, 9 days, 10 days to about 60 days, or from about 10 days to about 50 days, or from about 10 days to about 40 days, or from about 10 days to about 30 days, or from about 10 days to about 20 days. In another embodiment, where multiple dosing sessions are employed, the dosing sessions are spaced apart by from about 20 days to about 60 days, or from about 20 days to about 50 days, or from about 20 days to about 40 days, or from about 20 days to about 30 days. In even another embodiment, the multiple dosing sessions are bi-weekly (e.g., about every 14 days), monthly (e.g., about every 30 days), or bi-monthly (e.g., about every 60 days). In yet another embodiment, the dosing sessions are spaced apart by about 28 days. In an exemplary embodiment, the dosing sessions are spaced apart by 7 days.

### Co-Adminicraoon

In some embodiments, the immunoconjugate dimers described herein are administered in a co-therapeutic regimen to treat a patient for any one or more of the diseases or disorders associated with neovascularization mentioned herein. The method involves (either concurrent or non-concurrent) administration of a second active agent. In one embodiment, the second active agent is administered in the same composition as the immunoconjugate dimer. However, in another embodiment, the immunoconjugate dimer is administered in a separate composition. In one embodiment, the second active agent is a neovascularization inhibitor, an angiogenesis inhibitor, or a cancer chemotherapeutic. In one embodiment, the second active agent is a checkpoint inhibitor (anti-CTLA4, anti-PD1/PDL1). In another embodiment, the second active agent is an immunotherapy.

In one embodiment, the second active agent which is an angiogenesis or neovascularization inhibitor is a vascular endothelial growth factor (VEGF) inhibitor, a VEGF receptor inhibitor, a platelet derived growth factor (PDGF) inhibitor or a PDGF receptor inhibitor.

In another embodiment, the second active agent which is a neovascularization inhibitor is an integrin antagonist, a selectin antagonist, an adhesion molecule antagonist (e.g., antagonist of intercellular adhesion molecule (ICAM)-1, ICAM-2, ICAM-3, platelet endothelial adhesion molecule (PCAM), vascular cell adhesion molecule (VCAM)), lymphocyte function-associated antigen 1 (LFA-1)), a basic fibroblast growth factor antagonist, a vascular endothelial growth factor (VEGF) modulator, or a platelet derived growth factor (PDGF) modulator (e.g., a PDGF antagonist). In one embodiment of determining whether a subject is likely to respond to an integrin antagonist, the integrin antagonist is a small molecule integrin antagonist, for example, an antagonist described by Paolillo et al. (Mini Rev Med Chem, 2009, volume 12, pp. 1439-1446, incorporated by reference in its entirety), or a leukocyte adhesion-inducing cytokine or growth factor antagonist (e.g., tumor necrosis factor-α (TNF-α), interleukin-1β (IL-1β), monocyte chemotactic protein-1 (MCP-1) and a vascular endothelial growth factor (VEGF)), as described in U.S. Pat. No. 6,524,581, incorporated by reference in its entirety herein.

In another embodiment, the second active agent which is a neovascularization inhibitor is one or more of the following angiogenesis inhibitors: interferon gamma 1β, interferon gamma 1β (Actimmune®) with pirfenidone. ACUHTR028, αVβ5, aminobenzoate potassium, amyloid P, ANG1122, ANG1170, ANG3062, ANG3281, ANG3298, ANG4011, anti-CTGF RNAi, Aplidin, astragalus membranaceus extract with salvia and schisandra chinensis, atherosclerotic plaque blocker, Azol, AZX100, BB3, connective tissue growth factor antibody, CT140, danazol, Esbriet, EXC001, EXC002, EXC003, EXC004, EXC005, F647, FG3019, Fibrocorin, Follistatin, FT011, a galectin-3 inhibitor, GKT137831, GMCT01, GMCT02, GRMD01, GRMD02, GRN510, Heberon Alfa R, interferon α-2β, ITMN520, JKB119, JKB121, JKB122, KRX168. LPA1 receptor antagonist, MGN4220, MIA2, microRNA 29a oligonucleotide, MMI0100, noscapine, PBI4050, PBI4419, PDGFR inhibitor, PF-06473871, PGN0052, Pirespa, Pirfenex, pirfenidone, plitidepsin, PRM151, Px102, PYN17, PYN22 with PYN17, Relivergen, rhPTX2 fusion protein, RXI109, secretin, STX100, TGF-β Inhibitor, transforming growth factor, β-receptor 2 oligonucleotide, VA999260, XV615, or a combination thereof.

In another embodiment, the second active agent which is a neovascularization inhibitor is an endogenous angiogenesis inhibitors. In a further embodiment, the endogenous angiogenesis inhibitor is endostatin, a 20 kDa C-terminal fragment derived from type XVIII collagen, angiostatin (a 38 kDa fragment of plasmin), or a member of the thrombospondin (TSP) family of proteins. In a further embodiment, the angiogenesis inhibitor is a TSP-1, TSP-2, TSP-3, TSP-4 and TSP-5. Methods for determining the likelihood of response to one or more of the following angiogenesis inhibitors are also provided a soluble VEGF receptor, e.g., soluble VEGFR-1 and neuropilin 1 (NPR1), angiopoietin-1, angiopoietin-2, vasostatin, calreticulin, platelet factor-4, a tissue inhibitor of metalloproteinase (TIMP) (e.g., TIMP1, TIMP2, TIMP3, TIMP4), cartilage-derived angiogenesis inhibitor (e.g., peptide troponin I and chrondomodulin I), a disintegrin and metalloproteinase with thrombospondin motif 1, an interferon (IFN) (e.g., IFN-α, IFN-β, IFN-γ), a chemokine, e.g., a chemokine having the C—X—C motif (e.g., CXCL 10, also known as interferon gamma-induced protein 10 or small inducible cvtokine B10), an interleukin cytokine (e.g., IL-4, IL-12, IL-18), prothrombin, antithrombin III fragment, prolactin, the protein encoded by the TNFSF15 gene, osteopontin, maspin, canstatin, proliferin-related protein.

In one embodiment, one or more of the following neovascularization inhibitors is administered with the immunoconjugate described herein: angiopoietin-1, angiopoietin-2, angiostatin, endostatin, vasostatin, thrombospondin, calreticulin, platelet factor-4, TIMP, CDA1, interferon α, interferon β, vascular endothelial growth factor inhibitor (VEGI) meth-1, meth-2, prolactin, VEGI, SPARC, osteopontin, maspin, canstatin, proliferin-related protein (PRP), restin, TSP-1 TSP-2, interferon gamma 1β, ACUHTR028, αVβ5, aminobenzoate potassium, amyloid P, ANG1122, ANG1170, ANG3062, ANG3281, ANG3298, ANG4011, anti-CTGF RNAi, Aplidin, astragalus membranaceus extract with salvia and schisandra chinensis, atherosclerotic plaque blocker, Azol, AZX100, BB3, connective tissue growth factor antibody, CT140, danazol, Esbriet, EXC001, EXC002, EXC003, EXC004, EXC005, F647, FG3019, Fibrocorin, Follistatin, FT011, a galectin-3 inhibitor, GKT137831, GMCT01, GMCT02, GRMD01, GRMD02, GRN510, Heberon Alfa R, interferon α-2β, ITMN520, JKB119, JKB121, JKB122, KRX168, LPA1 receptor antagonist, MGN4220, MIA2, microRNA 29a oligonucleotide, MMI0100, noscapine, PBI4050, PBI4419, PDGFR inhibitor, PF-06473871, PGN0052, Pirespa, Pirfenex, pirfenidone, plitidepsin, PRM151, Px102, PYN17, PYN22 with PYN17, Relivergen, rhPTX2 fusion protein, RXI109, secretin, STX100, TGF-β Inhibitor, transforming growth factor, β-receptor 2 oligonucleotide, VA999260, XV615 or a combination thereof.

Yet another co-therapy embodiment includes administration of one of the immunoconjugates described herein with one or more of the following: pazopanib (Votrient), sunitinib (Sutent), sorafenib (Nexavar), axitinib (Inlyta), ponatinib (Iclusig), vandetanib (Caprelsa), cabozantinib (Cometrig), bevacizumab (Avastin), ramucirumab (Cyramza), regorafenib (Stivarga), ziv-aflibercept (Zaltrap), or a combination thereof. In yet another embodiment, the angiogenesis inhibitor is a VEGF inhibitor. In a further embodiment, the VEGF inhibitor is axitinib, cabozantinib, aflibercept, brivanib, tivozanib, ramucirumab or motesanib.

In one embodiment, the angiogenesis inhibitor is ranibizumab or bevacizumab. In a further embodiment, the angiogenesis in inhibitor is ranibizumab. In even a further embodiment, ranibizumab is administered at a dosage of 0.5 mg or 0.3 mg per dosing session, and is administered as indicated in the prescribing information for LUCENTIS.

In one embodiment, the co-therapy comprises administration of an antagonist of a member of the platelet derived growth factor (PDGF) family, for example, a drug that inhibits, reduces or modulates the signaling and/or activity of PDGF-receptors (PDGFR). For example, the PDGF antagonist, in one embodiment, is an anti-PDGF aptamer, an anti-PDGF antibody or fragment thereof, an anti-PDGFR antibody or fragment thereof, or a small molecule antagonist. In one embodiment, the PDGF antagonist is an antagonist of the PDGFR-α or PDGFR-β. In one embodiment, the PDGF antagonist is the anti-PDGF-β aptamer E10030, sunitinib, axitinib, sorefenib, imatinib, imatinib mesylate, nintedanib, pazopanib HCl, ponatinib, MK-2461, dovitinib, pazopanib, crenolanib, PP-121, telatinib, imatinib, KRN 633, CP 673451, TSU-68, Ki8751, amuvatinib, tivozanib, masitinib, motesanib diphosphate, dovitinib dilactic acid, linifanib (ABT-869).

### Pharmaceutical Compositions

The present application provides pharmaceutical compositions comprising any one of the immunoconjugate dimers described herein with one or more pharmaceutically acceptable excipients. In some embodiments the composition is sterile. The pharmaceutical compositions generally comprise an effective amount of the immunoconjugate dimer.

### Kits and Articles of Manufacture

The present application provides kits comprising an immunoconjugate dimer described herein. In some embodiments, the kits further contain a pharmaceutically acceptable excipient and instruction manual. In one specific embodiment, the kit comprises any one or more of the therapeutic compositions described herein, with one or more pharmaceutically acceptable excipients. The present application also provides articles of manufacture comprising any one of the therapeutic compositions or kits described herein. Examples of an article of manufacture include vials (including sealed vials).

The invention will be more fully understood by reference to the following examples. They should not, however, be construed as limiting the scope of the invention. It is understood that the examples and embodiments described herein are for illustrative purposes only.

### EXAMPLES

### Example 1—Evaluation of hI-Con1 in In Vitro Thrombin Generation Assays

The effect of hI-con1 (SEQ ID NO:2) in a thrombin generation assay in plasma was tested. Specifically, the effect of hI-con1 on thrombin generation in plasma in a tissue factor initiated reaction using a thrombogram (CAT-like) assay (Hemker et al. 2002. Pathophysiol. Haemost. Thromb. 32, pp. 249-253; Mann et al. 2007. J. Thromb Haemost. 5, pp. 2055-2061, each incorporated by reference herein in its entirety for all purposes) was evaluated. For the CAT-like assays, multidonor human citrate plasma from healthy individuals, human FVII-deficient plasma and normal rabbit citrate plasma were used. Thrombin (also referred to as Factor IIa, or activated blood coagulation factor II) generation was initiated either with human relipidated TF (in human plasma) or with rabbit relipidated TF (in rabbit plasma). hI-con1 was maintained frozen at −70° C. until use. Each sample included 3.0 mg hI-con1/mL in formulation buffer (15 mM HEPES, 150 mM NaCl, 5 mM CaCl₂,25 mM Arginine, 0.01%0/Tween 80, pH 7.4).

Human plasma FVIIa in 50% glycerol was purchased from Haematologic Technologies, Inc., 57 River Road, Essex Junction, Vt. 05452. It was stored at −20° C. until use. Before use, it was diluted to 10 nM in the formulation buffer (15 mM HEPES, 150 mM NaCl, 5 mM CaCl2), 25 mM Arginine, 0.01% Tween 80, pH 7.4).

Spectrozyme FXa (#222), lipidated recombinant human TF reagent (Catalog #4500L) and lipidated recombinant rabbit TF were purchased from American Diagnostica. Inc. (Stamford, Conn.), pooled normal human plasma (Lot #IR 11-020711) and rabbit plasma (Lot #26731) were purchased from Innovative Research Novi, Mich. 48377), congenital FVII-deficient plasma (Catalog #0700) was purchased from George King Bio-Medical, Inc. (Overland Park, Kans.) and human factor X (hFX) (#HCX-0050) and Phe-Pro-Arg-chloromethylketone (FPRck; Catalog #FPRCK-01), corn trypsin inhibitor (CTI; Catalog #CTI-01) were purchased from Haematologic Technologies, Inc (Essex Junction, Vt., USA). Fluorogenic substrate Z-Gly-Gly-Arg-AMC.HCl was purchased from Bachem (Torrance, Calif.) and ethylenediaminetetraacetic acid disodium salt dihydrate (EDTA; #E5134), NaCl (#S7653) and HEPES (#H3375) were purchased from Sigma (St. Louis, Mo.). HBS buffer, pH 7.4 contained 150 mM NaCl, 2 mM CaCh and 20 mM HEPES.

Active site inhibited FVIIa (FVIIai) was produced in house. 1, 2-Dioleolyl-sn-Glycero-3-Phospho-L-Serine (PS; #840035) and 1, 2-DioleoyJ-sn-Glycero-3-Phosphocholine (PC; #850375) were purchased from Avanti Polar Lipids, Inc. (Alabaster, Ala., USA). Phospholipid vesicles (PCPS) composed of 25% PS and 75% PC were prepared as described in Higgins and Mann 1983, incorporated by reference herein in its entirety for all purposes.

### Extrinsic FXase

Lipidated recombinant human TF (0.1 nM) was incubated with either 5 nM plasma FVIIa or 5 nM hI-con1 or mixture of both (each at 5 nM) and 100 μM PCPS for 10 min at 37° C. FX (4 μM) was added and at selected time points (0-5 min.) 10 μL aliquots of the reaction mixture were quenched into 170 μL HBS-0.1% PEG-20 mM EDTA. Twenty μL of Spectrozyme FXa (0.2 mM) was added and the rate of substrate hydrolysis was measured as an increase in absorbance at 405 nm (mOD/min).

### Thrombin Generation (CAT-Like) Assay

Corn trypsin inhibitor (CTI) at a final 0.1 mg/mL concentration was added to citrate plasma and 80 μL of this plasma was transferred into Immulon® 96-well plate (Thermo Electron Co., Waltham Mass.). When desired, hI-con1, plasma FVIIa and FVIIai were added at selected concentrations. Twenty μL of 5 pM TF and 20 μM PCPS mixture (both concentrations final) were added to CTI-plasma and incubated for 3 min. Thrombin generation was initiated by the addition of 20 μL of 2.5 mM ZGly-Gly-ArgAMC.HCl in HBS containing 0.1 M CaCl₂. Final concentration of substrate was 416 μM and that of CaCl₂ was 15 mM. Thrombin generation curves were generated using Thrombinoscope BY software.

### Results

Comparison of hI-Con1 with Plasma FVIIa in the Extrinsic FXase

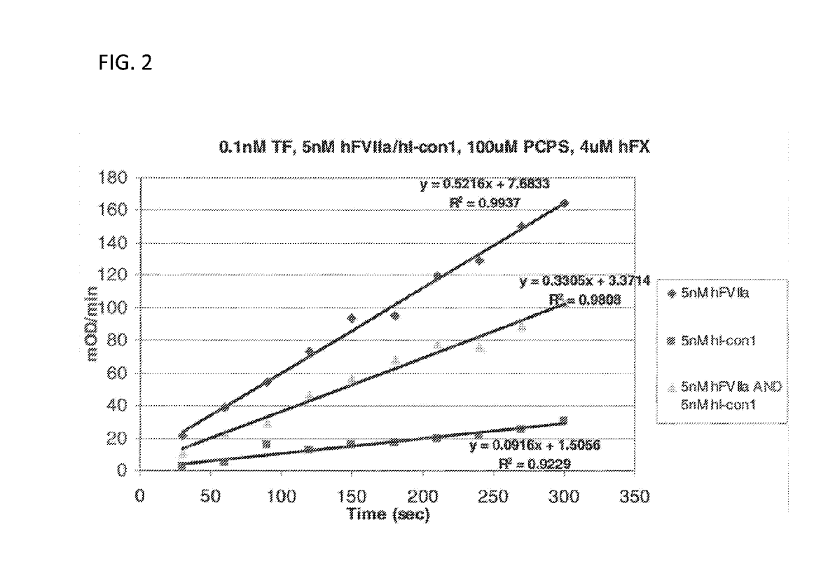

FXa-generating efficiency of two forms of FVIIa and of their mixture was determined in a chromogenic assay. hI-con1 was less active than plasma FVIIa. Activity of hI-con1 was 18% of that observed for plasma FVIIa. When both proteins were added at equimolar (5 nM) concentration, the rate of FXa generation in the middle between the rates observed for individual proteins, indicating that hI-con1 competes with plasma FVIIa for the limited amount of TF (FIG. 2). These data also suggest that hI-con1 has similar affinity for TF as plasma FVIIa.

### Thrombin Generation in Normal Human Plasma: The Effect of FVIIai

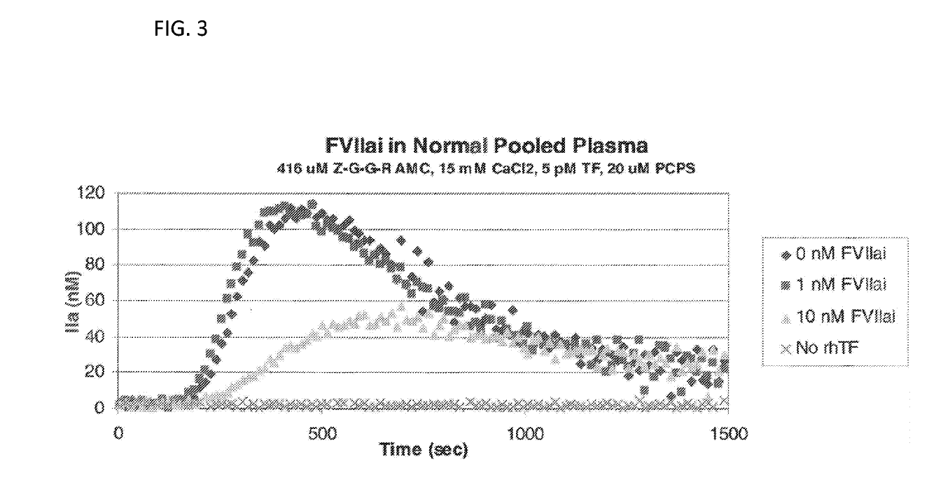

It was hypothesized that due to the low activity of the hI-con1 tissue factor (TF) complex in the extrinsic FXase, hI-con1 could act as an inhibitor by binding TF into an inefficient complex and preventing formation of an efficient complex between plasma FVIIa and TF. To test this hypothesis, the effect of a known inhibitor of coagulation. i.e., active site inhibited FVIIa (Kjalke et al. 1997), on thrombin generation in normal human plasma was evaluated. FVIIai at 1 nM concentration had no effect on thrombin generation initiated with lipidated human TF (FIG. 3). However at 10 nM, FVIIai prolonged the lag phase of thrombin generation and significantly suppressed both the maximum rate of thrombin generation and the maximum levels of thrombin produced. No thrombin generation was observed in the absence of TF.

Thrombin Generation in Normal Human Plasma: The Effect of hI-Con1

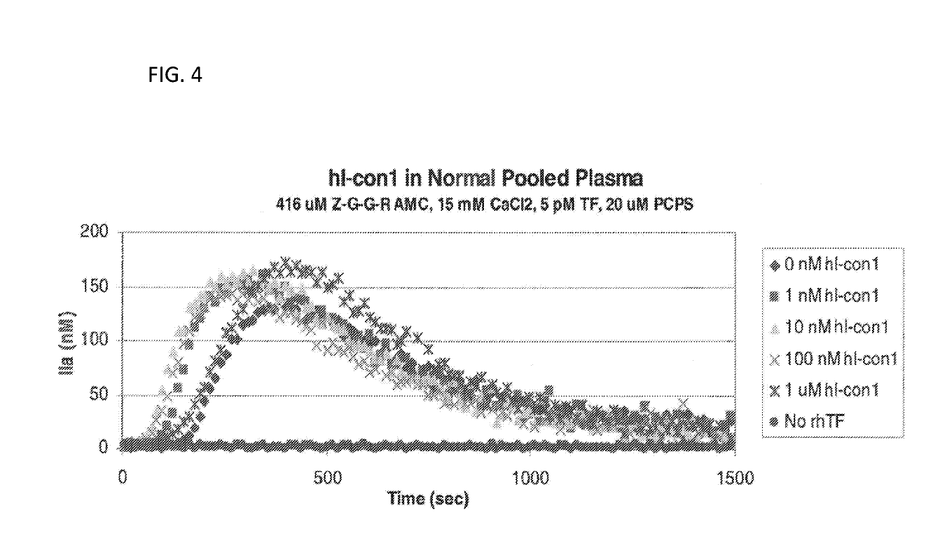

hI-con1 was titrated into normal human plasma initiated with TF to generate thrombin. Varying concentrations of hI-con1 was used, however even at extremely high hI-con1 concentrations (1 μM), no inhibition of thrombin generation was observed (FIG. 4).

### Thrombin Generation in Congenital FVII-Deficient Human Plasma

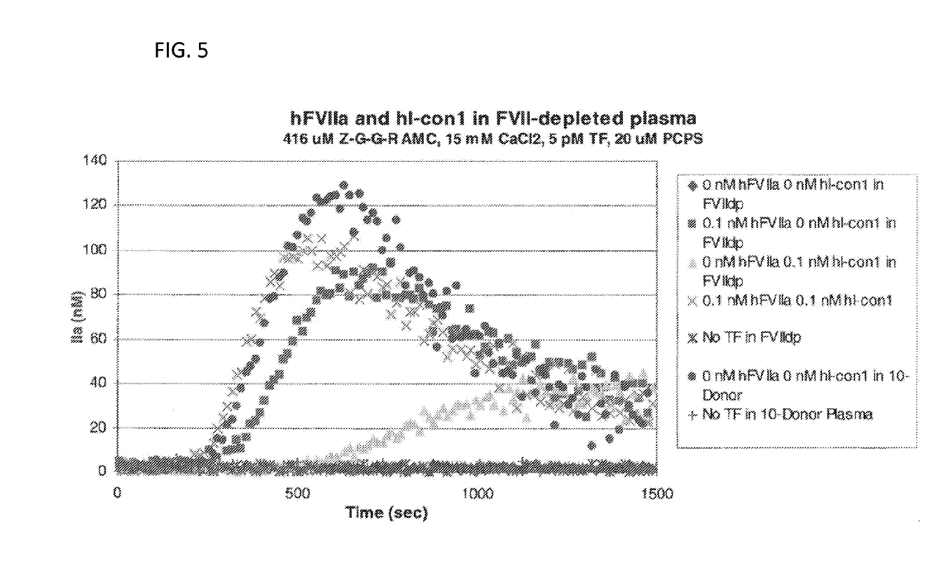

No thrombin generation was observed upon the addition of lipidated human TF to congenital FVII-deficient plasma, indicating that there no detectable functional FVIIa in that plasma (FIG. 5). An addition of 0.1 nM plasma FVIIa together with TF produced thrombin generation profile slightly lower than that observed in normal human plasma. An addition of 0.1 nM hI-con1 alone in the presence of TF led to the initiation of thrombin generation, however the process was significantly delayed and suppressed (FIG. 5). This result was consistent with the observation of low hI-con1 activity in the extrinsic FXase. The addition of both plasma FVIIa and hI-con1 at equimolar concentrations (0.1 nM) did not impair thrombin generation initiated with plasma FVIIa alone.

### Thrombin Generation in Normal Rabbit Plasma

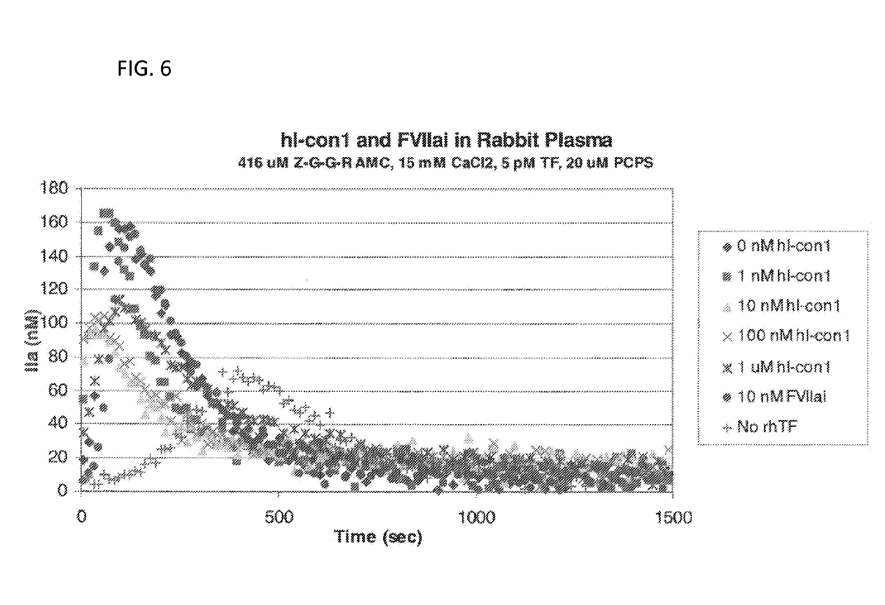

Thrombin generation in rabbit plasma was initiated with lipidated rabbit TF. The addition of 1 nM hI-con1 to this plasma had no pronounced effect on thrombin generation (FIG. 6). Similarly, no pronounced effect was observed when 10 nM FVIIai was added. At higher hI-con1 concentrations (10-1000 nM) some suppression in thrombin generation was observed. However the control experiment with no TF added led to thrombin generation, suggesting an endogenous presence of TF.

### Thrombin Generation in Centrifuged Rabbit Plasma

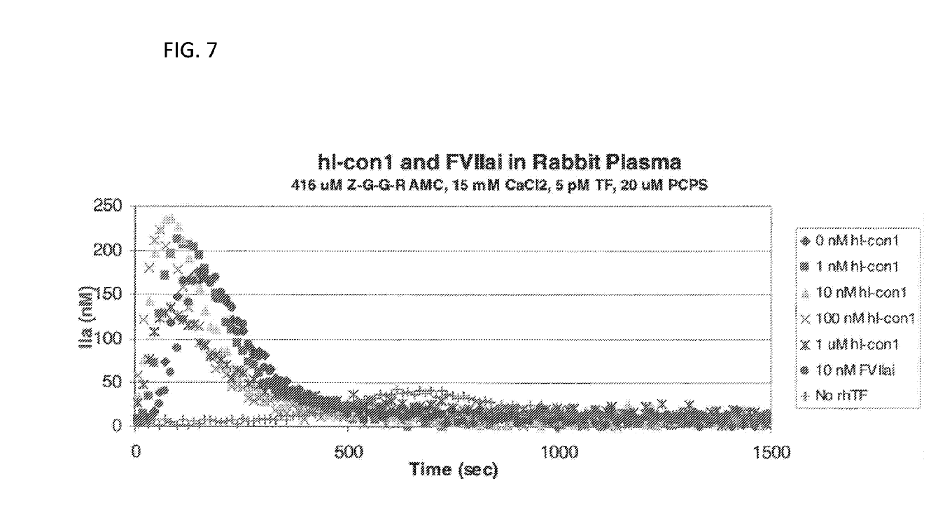

After centrifugation of rabbit plasma, an endogenous thrombin generating activity did not disappear completely, but was significantly decreased (FIG. 7). No suppression in TF-triggered thrombin generation was observed when 10 nM FVIIai was added. Similarly, no suppression was observed when 1-100 nM hI-con1 was added and only a limited decrease in thrombin generation was observed when high concentration (1 μM) hI-con1 was added (FIG. 7). These data indicate that at physiologically-relevant concentrations hI-con1 does not compete for rabbit TF with rabbit FVIIa.

### CONCLUSIONS

hI-con1 does not compete with plasma FVIIa for TF in the citrate plasma environment. hI-con1 has no pronounced (if any) effect on thrombin generation either in human plasma initiated with human TF or in rabbit plasma initiated with rabbit TF. It is not likely that hI-con1 would cause bleeding or thrombotic complications.

### Example 2—Effects of Treatment With hI-con1 on Choroidal Neovascularization in Pig

In this study, hI-con1 activity in a porcine wet AMD model (Kiilgaard et al., 2005. Acta. Ophthalmol. Scand. 83, pp. 697-704, incorporated by reference herein in its entirety for all purposes) and the optimal dose for the activity was examined. Additionally, the safety of hI-con1 when administered by intravitreal injection was determined.

In this study intravitreal injection of hI-con1 was demonstrated to result in the destruction of established laser-induced CNV in this porcine model. The injections of hI-con1 were well tolerated and the effects were dose-related, with and ED50 of 13.5 μg/dose. A major breakdown product of hI-con1 (100 kDa) was tested and was also well-tolerated and effective with an ED50 of 16.2 μg/dose.

### Test Articles

hI-con1 and 100 kD Fragment of hI-con1

hI-con1 was provided by Laureate Pharma Inc., 201 E. College Ave, Princeton, N.J., 08540. hI-con1 was maintained frozen at −70° C. until use: Lot PURIC1 080402 (SEC Fr 10-14), two vials each containing 200 μL at 2.0 mg/mL, 1.0 mg/mL, 0.5 mg/mL and 0.25 mg/mL in formulation buffer (15 mM HEPES, 150 mM NaCl, 5 mM CaCl2, 25 mM Arginine, 0.01% Tween 80, pH 7.4).

The following samples of the 100 kD fragment of hI-con1 were provided by Laureate Pharma Inc. 201 E. College Ave. Princeton, N.J., 08540. The fragment was maintained frozen at −70° C. until use: Lot PURIC1 080402 (SEC Fr 15), two vials each containing 200 μL at 2.0 mg/mL, 1.0 mg/mL, 0.5 mg/mL and 0.25 mg/mL in formulation buffer (15 mM HEPES, 150 mM NaCl, 5 mM CaCl2, 25 mM Arginine, 0.01% Tween 80, pH 7.4).

### Control Article

The formulation buffer (15 mM HEPES, 150 mM NaCl, 5 mM CaCl2, 25 mM Arginine, 0.01% Tween 80, pH 7.4) was used as the vehicle control.

### Test Animals

Two studies were conducted, each with groups of five (one group per test article) Yucatan miniature pigs (Sus scrofa), 10-12 weeks old, each weighting approximately 20 kilograms were bought from Professional Veterinary Research (Brownstown, Ind., USA).

### Husbandry

Each pig was maintained in a separate cage within a communal environment that housed four pigs. The lighting was computer controlled and set for a 6 am to 6 μm cycle. The temperature average was 70-72° F., with a variation of +/−1 degree. Humidity was kept between 30 and 70%, with average humidity equal to 33%. The animals were evaluated by the large animal husbandry supervisor and a licensed veterinary technician on arrival and a licensed veterinary technician once weekly until they were euthanized. A veterinarian evaluated the animals to determine if there were any abnormalities or concerns. The animals were quarantined for about 1 week prior to experiments.

### Feed and Water

Daily feed and water were provided to the miniature pigs. They were bedded on hay that served as a feed supplement. The feed was Purina #5084, Laboratory Porcine Grower Diet, Manufactured by Purina Mills, LLC, 555 Maryville University Drive. Suite 500, St. Louis, Mo. 63141, and fed at 2% body weight per day. The water was 0.5 micron filtered tap water. It was not routinely analyzed for contaminants except by the water company and reports are reviewed annually.

### Justification of Species

hI-con1 has limited cross-species activity and the pig is one of the few laboratory animal species in which it is active. The vitreous cavity of the pig is approximately 3 mL, allowing intravitreal injection of reasonable volumes of test article. The pig eye has retinal vascular similarities to humans in addition to several cone-dominant regions of the retina that are similar to the human macula.

### Methods

### Laser-Induced Choroidal Neovascularization

Under general anesthesia, the pupils of the animals were dilated with 1% tropicamide and 2.5% phenylephrine. An indirect ophthalmoscope with a double-frequency YAG laser (532 nm) was used to deliver 74 spots per eye using a 2.2 D lens and the following laser parameters: laser power 1000-1500 mW, duration 0.1 seconds, and repetition rate 500 msec. The laser treatment was designed to yield a microrupture of the Bruch's membrane, generating CNV at 60-70% of the laser spots within two weeks (Bora et al., 2003, incorporated by reference herein in its entirety for all purposes).

### Study Design

The study design is summarized in Table 4 below.

TABLE 4
Study design.

| | |
|---|---|
| Pig | Dose injected (μg) in each eye |

| Number | hI-con1 | hI-con1 100 kD fragment |
|---|---|---|
| 1 | 0 | 0 |
| 2 | 25 | 25 |
| 3 | 50 | 50 |
| 4 | 100 | 100 |
| 5 | 200 | 200 |

Choroidal neovascularization was induced on Day 0 in both eyes of two groups of 5 pigs. On Day 10, 100 μL of solutions of hI-con1 (Study 1) or its 100 kD fragment (Study 2) at 0.25, 0.5, 1.0 or 2.0 mg/mL were administered by intravitreal injection into both eyes of the pigs as shown in Table 4. On Day 10, 100 μL of formulation buffer was administered by intravitreal injection into both eyes of the control pigs.

### Test and Control Articles Administration

The animals were anesthetized with a mixture of ketamine hydrochloride (40 mg/kg) and xylazine hydrochloride (10 mg/kg). Injections were administered using a strict sterile technique, which involved scrubbing the lids with a 5% povidone-iodine solution and covering the field with a sterile eye drape. A sterile lid speculum was used to maintain exposure of the injection site. All injections were performed 2 mm from the limbus through the pars plana, using a 30-gauge needle on a 1 mL tuberculin syringe. After injection, a drop of 2% cyclopentolate and antibiotic ointment was placed in the eye. The animals were examined daily for signs of conjunctival injection, increased intraocular pressure, anterior uveitis vitritis, or endophthalmitis, and were sacrificed on Day 14.

### Terminal Procedures

On Day 14, the pigs were anesthetized with an 8:1 mixture of ketamine and xylazine and perfused through the ear vein with 10 mL PBS containing 3 mg/mL fluorescein-labeled dextran with an average molecular weight 2×10⁶ (Sigma. St. Louis, Mo., USA). The eyes were enucleated and four stab incisions were made at the pars plana followed by fixation in 4% paraformaldehyde for 12 hours at 4° C. The cornea and the lens were removed, and the neurosensory retina was dissected from the eyecup and four radial cuts were made from the edge of the eyecup to the equator. The choroid-retinal pigment epithelium (RPE) complex was separated from the sclera and flatmounted on a glass slide in Aquamount with the inner surface (RPE) facing upwards. Flat mounts were stained with a monoclonal antibody against elastin (Sigma) and a Cy3-conjugated secondary antibody (Sigma) and examined with a confocal microscope (Zeiss LSM510, Thornwood, N.Y., USA). The vasculature, filled with dextranconjugated fluorescein, stained green and the elastin in the Bruch's membrane stained red. The level of the Bruch's membrane was determined by confocal microscopy using the intense red signal within a series of z-stack images collected at and around the laser spot. The presence of CNV was indicated by the branching linear green signals above the plane of Bruch's membrane. Absence of CNV was defined under very stringent criteria as the total absence of green fluorescence in the vessels in the spot (Tezel, 2007. Ocular Imm Inflamm 15, pp. 3-10.

### Statistical Analysis

The percentage of laser spots with CNV at different doses of hI-con1 or its 100 kD fragment was compared pair wise by a chi-square test. The results were plotted against the hI-con1 dose to derive the best-fit curve, which was used to calculate the dose of hI-con1 that reduces the fraction of laser spots with CNV by 50% (ED50). A confidence level of p<0.05 was considered to be statistically significant.

### Results

Effects of Intravitreal Treatment with hI-con1 on CNV

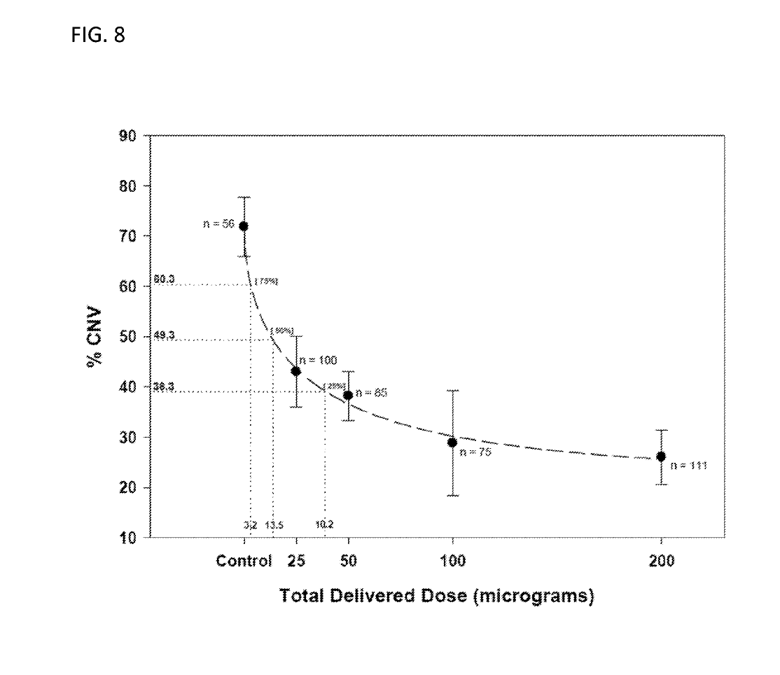

Choroidal neovascularization developed in 71.9±5.8% of the laser spots in control eyes. A single intravitreal injection of hI-con1 on Day 10 in pig eyes (n=2 at each dose) significantly reduced subretinal CNV on Day 14 at all doses tested, i.e., 25-200 μg, Table 5: FIG. 8). The inhibitory effect of hI-con1 fit well to a 5-parameter Sigmoidal Weibull curve. The dose causing a 50% decrease in the yield of CNV (ED50) was 13.5 μg.

TABLE 5
Effects of intravitreal treatment with hI-con1 on
CNV incidence in laser-induced CNV pig model.

| | Laser Spots | | | | |
|---|---|---|---|---|---|
| Pig | Examined | | hI-con1 dose | % spots with CNV | P value |
| Number | Left | Right | (μg) | (Average ± SD) | (vs. control) |
| 1 | 25 | 31 | 0 | 71.9 ± 5.8 | |
| 2 | 50 | 50 | 25 | 43.0 ± 7.1 | 0.001 |
| 3 | 36 | 49 | 50 | 38.2 ± 4.9 | <0.001 |
| 4 | 47 | 28 | 100 | 28.8 ± 10.4 | <0.001 |
| 5 | 54 | 57 | 200 | 26.0 ± 5.4 | <0.001 |

Effects of Intravitreal Treatment with 100 kD fragment of h-con1 on CNV

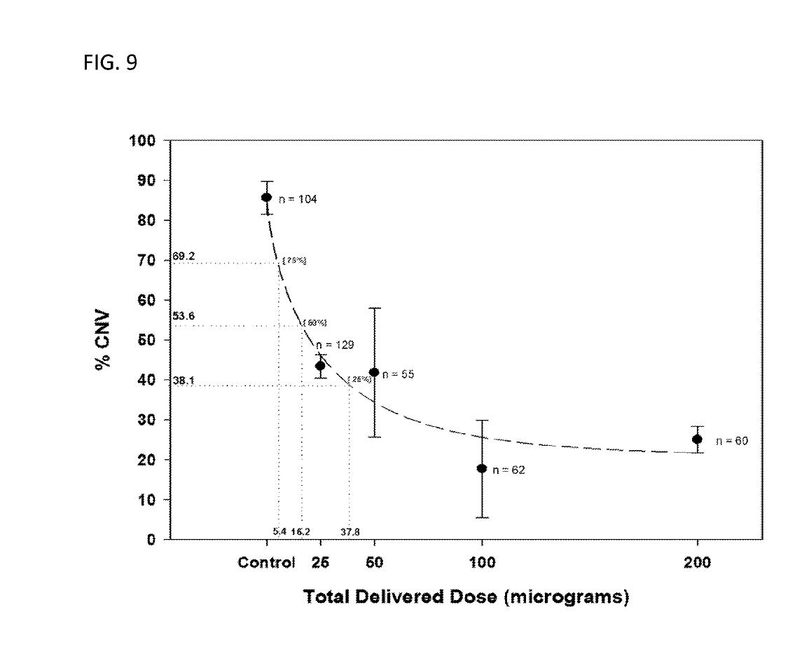

Choroidal neovascularization developed in 85.6±4.1% of the laser spots in control eyes. A single intravitreal injection of the 100 kDa fragment of hI-con1 on Day 10 in pig eyes (n=2 at each dose) significantly reduced subretinal CNV on Day 14 at all doses tested, i.e., 25-200 μg, Table 6. FIG. 9). The inhibitory effect of hI-con1 fit well to a 5-parameter Sigmoidal Weibull curve. The dose causing a 50% decrease in the yield of CNV (ED50) was 16.2 μg.

TABLE 6
Effects of intravitreal treatment with 100 kD fragment of
hI-con1 on CNV incidence in laser-induced CNV pig model.

| | Laser Spots | | | | |
|---|---|---|---|---|---|
| Pig | Examined | | hI-con1 dose | % spots with CNV | P value |
| Number | Left | Right | (μg) | (Average ± SD) | (vs. control) |
| 1 | 52 | 52 | 0 | 85.6 ± 4.1 | |
| 2 | 73 | 56 | 25 | 43.4 ± 2.9 | <0.001 |
| 3 | 26 | 29 | 50 | 41.8 ± 16.1 | <0.001 |
| 4 | 30 | 32 | 100 | 17.7 ± 12.2 | <0.001 |
| 5 | 14 | 46 | 200 | 25.0 ± 3.3 | <0.001 |

Intravitreal injection of hI-con1 and its 100 kD fragment at doses from 25-200 μg caused significant regression of pre-existing laser-induced CNV 4 days after the injections were administered. The response of the lesions to the injections was clearly dose-related with ED50 doses of 13.5 and 16.2 μg, respectively. These results indicate that the specific activity of the 100 kD fragment of hI-con1 is similar to that of the intact molecule. Doses greater than 100 μg had very little additional decrease in CNV; thus, the efficacious dose in this model is ≤100 μg.

### Example 3—Tissue Cross-Reactivity Study of hI-con1 with Normal Human Tissues

In this study, the binding of hI-con1 to normal human tissues was assessed using standard immunohistochemistry (IHC) techniques, in a standard tissue cross-reactivity (TCR) study. The study was performed utilizing a single batch of biotinylated hI-con1 for IHC staining of normal, as well as positive and negative control human tissues. A positive staining result is indicative of potential toxicities associated with administration of hI-con1 to humans in vivo.

In this model, tissue staining was observed only in the positive control colon carcinoma tumor. All other normal human tissues showed no immunoreactivity. These findings indicate that hI-con1 binding is specific to abnormal tissue, with no binding to normal tissues observed.

### Example 4—Evaluation of the Binding of hI-con1 to Lipidated Tissue Factors

To allow for cross species comparison, a Biacore study of the kinetics of binding of hI-con1 and hFVIIa to human lapidated TF (hTF) and rabbit lipidated TF (rTF) was conducted.

As described in detail below, hI-con1 and hFVIIa both bound with high and approximately equal affinity to lapidated hTF.

### Materials and Methods

Lipidated rabbit tissue factor (rTF; Product #4520L; Lot #051017) purchased from American Diagnostica. Lipidated human tissue factor (hTF; Lot FIL105HO1) supplied by Main Biological Laboratories, 378 Bel Marin Keys, Novato, Calif. 94949.

hI-con1; 1 ml; 100 μg/ml; MW 157 kDa

Human FVIIa; Lot # A09050525 (Fitzgerald); 1.01 mg/ml; 40 μL/vial; MW 50 kDa

### Equipment: Biacore 3000; CM5 Sensor Chip

The GE procedure for proteoliposome immobilization (amine coupling) protocol was used to coat PS/PC/rTF on flow cell 2, and PS/PC/hTF on flow cell 3. Flow cells were equilibrated with running buffer (15 mM Hepes, 150 mM NaCl, 5 mM CaCl2), 25 mM Arginine, 0.01% Tween 80, pH7.4) at a flow rate of 5 μL/min. Kinetic analyses were performed at 37° C. by flowing consecutively increasing concentrations of each analyte (0-10 nM) in the running buffer (15 mM HEPES, 150 mM NaCl, 5 mM CaCl2, 25 mM Arginine, 0.01% Tween 80, pH 7.4) over the sensor chip for 5 min followed by a 10 min dissociation period at a flow rate of 30 μL/min in parallel.

Analyte binding to the lipidated TF was determined by subtracting the RU values noted in the reference flow cell 1 from flow cell 2 and 3. Binding of analytes to the TFs was monitored in real time to obtain on (ka) and off (kd) rates. The equilibrium dissociation constant (KD) was calculated from the observed ka and kd.

The chips were regenerated with 3 min pulses of 10 mM EDTA in HEPES buffer (20 mM HEPES, 150 mM NaCl, pH 7.4).

Capture of rTF to the chip—Flow cell 2 was coated with rabbit TF (>Resonance Units [RU]10,000) by amine coupling. Flow cell 3 was coated with human TF (>RU 8,000) by amine coupling.

### Determination of the Amount of Test Ligands (RL) to be Captured on the Chip

In this experiment, the desired level of R_{Max} for the measurement of ligand-analyte interaction was based on the value determined by a previous experiment where rTF captured at 10,000 Resonance Units (“RU”) gave binding of hI-con1 with R_{Max} of 15 RU and hTF captured at 8,000 RU gave binding of hI-con1 with R_{Max} of 10RU. The amount of the analyte to be captured on the chip depended on the molecular weights of the interacting proteins. It is determined by the following formula:

R_{Max}=MW_{A}/MW_{L}·R_{L}

MW_{A} is the molecular weight of the analyte (157 kDa for hI-con1, 50 kDa for hFVIIa. and 150 kDa for IgG1). MW_{L} is the molecular weight of the ligand, in this assay it is expected to be very large (multiple of 35 kDa).

### Flow Rate of the Antibody Solution

The flow rate used for capturing the ligand was 10 μL/min. For kinetics analysis, the flow rate of 30 μL/min. was used.

### Kinetic Analysis

Based on the saturation concentration of the analyte, binding analysis was performed using saturating analyte concentrations of 0-500 nM for rabbit TF and 0-50 nM for human TF. Chi squared (χ²) analysis was carried out between the actual sensorgram and the calculated on- and off-rates to determine the accuracy of the analysis.

χ² value up to 2 is considered significant (accurate) and below 1 is highly significant (highly accurate).

The Biacore assay results are provided in Table 7 below.

TABLE 7
Biacore assay results

| | | | | | | | | | Chi |
|---|---|---|---|---|---|---|---|---|---|
| | | Ka | Dk | Rmax | Conc. of | | KA | K_{D} | squared |
| Ligand | Analyte | (1/Ms) | (1/s) | (RU) | analyte | | (1/M) | (M) | (χ²⁾ |
| Rabbit TF | hI-con1 | 5.6 × 10⁴ | 3.0 × 10⁻³ | 4.92 | 0-500 | nM | 1.9 × 10⁷ | 5.3 × 10⁻⁸ | 5.6 × 104 |
| Rabbit TF | hFVIIa | 3.4 × 10⁴ | 1.4 × 10⁻³ | 4.9 | 0-500 | nM | 2.5 × 10⁷ | 4.0 × 10⁻⁸ | 0.06 |
| Human TF | hI-con1 | 6.2 × 10⁵ | 5.5 × 10⁻⁴ | 71.5 | 0-50 | nM | 1.1 × 10⁹ | 8.9 × 10⁻¹⁰ | 3.34 |
| Human TF | hFVIIa | 5.1 × 10⁵ | 9.3 × 10⁻⁴ | 47 | 0-20 | nM | 5.5 × 10⁸ | 1.8 × 10⁻⁹ | 0.13 |

As shown in Table 8 below, hI-con1 and hFVIIa both bound with high, and approximately equal, affinity to lipidated hTF. Both ligands also bound to lipidated rTF with approximately 10-fold lower affinities.

TABLE 8
Relative affinities for the binding
of hI-con1 and hFVIIa to hTF and rTF

Dissociation Constant (K_{D})
Analyte

| Ligand | hI-con1 | hFVIIa |
|---|---|---|
| Human Tissue Factor | 8.92 × 10 ⁻¹⁰ M | 1.81 × 10⁻⁹ M |
| Rabbit Tissue Factor | 5.32 × 10 ⁻⁸ M | 3.95 × 10⁻⁸ M |

### Example 5—Randomized, Double-Masked, Multicenter, Active-Controlled Study Evaluating hI-con1 in Patients with CNV Secondary to Age-Related Macular Degeneration

In this study, the safety of intravitreal injections of hI-con1, administered as monotherapy or in combination with ranibizumab (LUCENTIS) compared to ranibizumab monotherapy in patients with choroidal neovascularization (CNV) secondary to age-related macular degeneration (AMD) is assessed. Additionally, the biological activity and pharmacodynamics effect of hI-con1, administered as monotherapy or in combination with ranibizumab (LUCENTIS) compared to ranibizumab monotherapy is assessed.

The study presented in this example is a randomized, double-masked, multicenter, active-controlled study. Patients enrolled in this study are naïve to treatment for CNV. Patients are randomly assigned to one of the following three treatment arms in the selected study eye in a 1:1:1 ratio:
hI-con1 monotherapy (0.3 mg)+sham injection
ranibizumab monotherapy (0.5 mg)+sham injection
hI-con1 (0.3 mg)+ranibizumab (0.5 mg) combination therapy

Randomization is stratified by best-corrected visual acuity (BCVA) letter score in the study eye at baseline (≤54 letters versus ≥55 letters) and by study site.

Patients receive up to two intravitreal injections at each injection visit. In order to maintain the study mask among the treatment arms, a sham injection is employed in patients receiving monotherapy.

Patients are administered intravitreal injections in the study eye once every four weeks at months 0, 1 and 2. As of Month 3 (at Months 3, 4 and 5) patients are retreated according to their assigned treatment arm, based on their individual observed treatment response. The masked Investigator uses the following retreatment criteria (based on the category of individual patient response) to determine if treatment is required at these visits:
Loss of ≥5 letters of BCVA due to AMD compared to the previous scheduled visit.
Independent of BCVA change, any anatomical evidenced of increased CNV activity (e.g., new or increased fluid and/or leakage, hemorrhage) compared to the previous visit.
No BCVA change compared to Baseline (Visit 2), but there is anatomical evidence of persistent CNV activity (e.g., same persistent fluid and CST compared to Baseline.

Rescue treatment with 0.5 mg of ranibizumab is administered to the study eye as an add-on therapy at any time during the 6-month treatment and follow-up period if either of the following conditions occur:
Loss of ≥15 letters of BCVA due to AMD compared to Baseline (Visit 2).
Loss of ≥10 letters from baseline (Visit 2) of BCVA due to AMD that is confirmed at two consecutive visits. Patients with a loss of ≥10 letters compared to baseline are requested to return within 7 days or as soon as possible for additional follow up at an unscheduled visit.

The masked physician makes the determination if rescue treatment is needed according to the above criteria. If rescue treatment is administered to the study eye during a scheduled injection visit, to ensure that the study masking is maintained, the unmasked physician administers rescue treatment and the patient's scheduled study treatment/re-treatment is as follows.
hI-con1 monotherapy arm: hI-con1 (0.3 mg)+rescue therapy (0.5 mg ranibizumab).
ranibizumab monotherapy arm: ranibizumab (0.5 mg)+sham injection.
combination therapy: hI-con1 (0.3 mg)+ranibizumab (0.5 mg).

If rescue treatment is administered to the study eye at an unscheduled visit, the unmasked physician administers rescue treatment as requested.

If rescue treatment is administered to the study eye, the patient continues with the study visit schedule for the next visit in accordance with the protocol and continues receiving study treatment according to the assigned randomization arm.

Safety is evaluated by tracking of adverse events, clinical laboratory tests (serum chemistry, hematology and coagulation), vital signs measurements, abbreviated physical examinations, slit-lamp biomicroscopy, intraocular pressure (IOP) and dilated ophthalmoscopy. Pharmacodynamic and biological activity is measured by means of BCVA by ETDRS visual acuity chart, spectral-domain optical coherence tomography (sdOCT), color fundus photography (CFP), fundus fluorescein angiography (FA), fundus autofluorescence (FAF), contrast sensititivy, and microperimetry. Pharmacokinetic (PK) and immunogenicity is evaluated by means of measuring plasma concentrations of hI-con1 and anti-drug antibodies.

### Example 6—Repeated Escalating Intravitreal Doses of hI-Con1 in Patients with Uveal Melanoma Who are Planned to Undergo Nucleation or Brachytherapy

This example describes a Phase 1, open-label, multicenter study evaluating the safety and tolerability, biologic activity, pharmacodynamics, and pharmacokinetics of single and repeated escalating intravitreal doses of hI-con1 in patients with uveal melanoma who are planned to undergo enucleation or brachytherapy.

One objective of this study is to evaluate the safety and tolerability of single and repeated escalating intravitreal injections of hI-con1 administered in patients with uveal melanoma who are planned to undergo enucleation or brachytherapy. This is determined by monitoring the occurrence of ocular and systemic adverse events. Changes in standard clinical laboratory tests are also monitoried (serum chemistry, hematology, coagulation) and ADA levels. Changes in vital signs are mointored, and physical and ophthalmic examinations are carried out.

Another objective is to assess the pharmacokinetics and pharmacodynamic effect of hI-con1 in patients with uveal melanoma. This objective includes measuring plasma levels of hI-con1 following intravitreal injections of hI-con1, measuring changes in circulating TF levels and serum cytokine levels following intravitreal injections of hI-con1; and characterization of immunophenotyping patterns.

Another objective is to describe preliminary evidence of the biologic activity of hI-con1 as determined by changes in visual acuity, and tumor size. This includes monitoring change in tumor size (thickness, volume) as assessed by imaging techniques (CFP, FA, sdOCT, ultrasound, ICG angiography, EDI-OCT) from baseline.

Another objective is to determine the prognostic indicator of the genetic profile of the tumor in relation to Tissue Factor expression and response to hI-con1 therapy. This includes the characterization of the genetic profile of the tumor (Type 1 or Type 2), and associated changes in the tumor TF with hI-con1 treatment. This includes the characterization of pathologic findings of the tumor (hI-con1 staining of tumor structures, TF and PAR2 expression, characterization of the immune infiltrate, and alterations of the vasculature). This also includes hI-con1 and proteomic analysis (TF and other cytokine levels) of the vitreous humor.

Another objective is to describe preliminary evidence of the biologic activity of hI-con1 as determined by changes in tumor pathology, and proteomic analysis of vitreous humor.

Patients diagnosed with primary uveal melanoma involving the posterior uveal tract for which the selected treatment management plan is enucleation or brachytherapy may be screened for this study. Eligible patients are assigned to one of three cohorts:

Cohort 1 (enucleation or brachytherapy): Single dose of 0.3 mg (300 μg/100 μl) hI-con1.
Cohort 2 (enucleation or brachytherapy): Two doses of 0.3 mg (300 μg/100 μl) hI-con1 each administered one week apart.
Cohort 3 (enucleation or brachytherapy): Two doses of 0.6 mg (300 μg/100 μl+300 μg/100 μl) hI-con1 each administered one week apart.

hI-con1 is administered on Day 0 for all cohorts, and in addition on Day 7 for cohorts 2 and 3 only. Patients return to the study center on Day 1 for a safety follow-up visit (all cohorts) and 1 day post Day 7 (cohorts 2 and 3 only). Patients return to the clinic for safety, clinical, and imaging assessments prior to the scheduled surgical procedure. Patients have their planned surgical procedure (enucleation or brachytherapy) as early as 4 days after the last hI-con1 treatment, and return to the study center 30 days post-procedure for follow-up safety assessments (and ocular assessments for brachytherapy patients) and end of study visit.

For enucleation patients: immediately following the enucleation procedure, vitreous humor is aspirated for intraocular hI-con1 and proteomic analysis of TF and cytokine levels. Following the clinical site's standard of care pathology of the tumor, prepared slides and/or tumor block samples are sent to a central laboratory for the study for additional study-related pathology evaluation of the tumor (including hI-con1 binding, TF expression, protease activating receptors 2 (PAR2) expression, immune infiltrate, and alterations of the vasculature), genetic profiling, and Exome sequencing of the tumor. Genetic profiling of the tumor previously performed with fine needle aspiration of the tumor does not require repeated testing.

hI-con1 is supplied in single-use glass vials containing 0.28 mL of a sterile solution of hI-con1 at a concentration of 3 mg/mL in 15 mM HEPES, 150 mM NaCl, 25 mM Arginine, pH 7.4 with 0.01% of Polysorbate-80 and 5 mM CaCl2.

While the described invention has been described with reference to the specific embodiments thereof it should be understood by those skilled in the art that various changes may be made and equivalents may be substituted without departing from the true spirit and scope of the invention. The various embodiments described above can be combined to provide further embodiments. In addition, many modifications may be made to adopt a particular situation, material, composition of matter, process, process step or steps, to the objective spirit and scope of the described invention. All such modifications are intended to be within the scope of the claims appended hereto.

1. An immunoconjugate dimer, wherein the monomer subunits of the dimer each comprises the sequence of SEQ ID NO: 11, SEQ ID NO: 12, SEQ ID NO: 13, or SEQ ID NO: 14.

2. An immunoconjugate dimer, wherein the monomer subunits of the dimer each comprises a mutated Factor VIIa (FVIIa) targeting domain conjugated to an immunoglobulin G1 (IgG1) Fc effector domain, wherein the targeting domain comprises the sequence of SEQ ID NO:17 or SEQ ID NO: 18, and wherein the effector domain comprises a hinge region and the effector domain comprises the sequence of SEQ ID NO:21, SEQ ID NO:22, SEQ ID NO:23, or SEQ ID NO:24.

3. A method of treating a disease or disorder associated with neovascularization in a patient in need thereof, comprising administering to the patient in one or more dosing sessions, a composition comprising an effective amount of the immunoconjugate dimer of any one of claims 1-2.

4. The method of claim 3, wherein the disease or disorder is atherosclerosis, rheumatoid arthritis, ocular melanoma, cancer, diabetic macular edema (DME), macular edema following retinal vein occlusion (RVO), proliferative diabetic retinopathy, wet age-related macular degeneration (AMD), retinopathy of prematurity (ROP), or neovascular glaucoma.

5. The method of claim 4, wherein the disease or disorder is ocular melanoma.

6. The method of claim 4, wherein the disease or disorder is wet AMD.

7. The method of claim 4, wherein the disease or disorder is cancer.

8. A method for treating ocular melanoma in a patient in need thereof, the method comprising administering to the patient in one or more dosing sessions, a composition comprising an effective amount of an immunoconjugate dimer, wherein the monomer subunits of the dimer each comprises a mutated Factor VIIa (FVIIa) protein conjugated to an immunoglobulin G1 (IgG1) Fc domain.

9. The method of claim 8, wherein the mutated Factor VIIa (FVIIa) protein is human.

10. The method of claim 8, wherein the immunoglobulin G1 (IgG1) Fc domain is human.

11. The method of claim 8, wherein treating the ocular melanoma comprises preventing, inhibiting, or reversing neovascularization in the eye of the patient in need of treatment.

12. The method of claim 11, wherein the neovascularization is choroidal neovascularization.

13. A method for preventing or slowing metastasis of an ocular melanoma in a patient in need thereof, comprising administering to the patient in one or more dosing sessions, a composition comprising an effective amount of an immunoconjugate dimer, wherein the monomer subunits of the dimer each comprises a mutated Factor VIIa (FVIIa) protein conjugated to an immunoglobulin G1 (IgG1) Fc domain.

14. A method for decreasing the size of an ocular melanoma tumor in an eye of a patient in need thereof, comprising, administering to the patient in one or more dosing sessions, a composition comprising an effective amount of an immunoconjugate dimer, wherein the monomer subunits of the dimer each comprises a mutated Factor VIIa (FVIIa) protein conjugated to an immunoglobulin G1 (IgG1) Fc domain.

15. The method of claim 13 or claim 14, wherein the mutated Factor VIIa (FVIIa) protein is human.

16. The method of claim 13 or claim 14, wherein the immunoglobulin G1 (IgG1) Fc domain is human.

17. The method of claim 14, wherein the tumor exhibits at least a 10% reduction in size post administration of the immunoconjugate, as measured by comparing the size of the tumor having been treated versus the size of the tumor prior to treatment.

18. The method of any one of claims 1-17, wherein the ocular melanoma is a uveal melanoma.

19. The method of claim 18, wherein the uveal melanoma is an anterior uveal tract melanoma.

20. The method of claim 19, wherein the anterior uveal tract melanoma is an iris melanoma.

21. The method of claim 18, wherein the uveal melanoma is a posterior uveal tract melanoma.

22. The method of claim 21, wherein the posterior uveal tract melanoma is a ciliary body melanoma.

23. The method of claim 21, wherein the posterior uveal tract melanoma is a choroid melanoma.

24. The method of any one of claims 1-17, wherein the immunoconjugate is administered in a dose of between 1 μg and 1500 μg in each of the one or more dosing sessions.

25. The method of claim 24, wherein the dose is selected from the group consisting of about 30 μg, about 150 μg, about 300 μg, and about 600 μg.

26. The method of claim 24, wherein the immunoconjugate is suspended in a volume of between 10 μL and 200 μL.

27. The method of claim 26, wherein the immunoconjugate is suspended in a volume of about 20 μL, about 50 μL, or about 100 μL.

28. The method of any one of claims 3-17, wherein administering comprises intravitreal or suprachoroidal injection.

29. The method of any one of claims 3-27, wherein administering comprises intravenous administration.

30. The method of any one of claims 3-27, wherein administering comprises intratumoral administration.

31. The method of claim 8, wherein treating ocular melanoma comprises slowing the onset of metastasis of the melanoma or prevention of metastasis of the melanoma in the eye of the patient in need of treatment.

32. The method of any one of claims 3-31, wherein the immunoconjugate dimer is a homodimer.

33. The method of any one of claims 3-31, wherein the immunoconjugate dimer is a heterodimer.

34. The method of claim 32 or 33, wherein at least one of the monomer subunits of the immunoconjugate comprises a mutated human FVIIa domain comprising a single point mutation at Lys341 or Ser344.

35. The method of claim 34, wherein the single point mutation is to an Ala residue.

36. The method of claim 35, wherein the single point mutation is Lys341 to Ala341.

37. The method of claim 35, wherein the single point mutation is Ser344 to Ala344.

38. The method of any one of claims 3-37, wherein said one or more dosing sessions comprise two or more, three or more, four or more, or five or more dosing sessions.

39. The method of any one of claims 3-38, wherein each dosing session is spaced apart by from about 20 days to about 50 days, or from about 20 days to about 40 days, or from about 20 days to about 30 days.

40. The method of any one of claims 3-39, wherein there are two dosing sessions, and each dosing session is spaced apart by about 7 days.

41. The method of any one of claim 38 or 40, wherein said one or more dosing sessions comprise 12 to 24 dosing sessions.

42. The method of any one of claims 8-41, wherein administering comprises intravitreal injection of the composition into the eye of the patient once every 28 days, once every 30 days, or once every 35 days.

43. The method of any one of claims 8-42, wherein the monomer subunits of the dimer each comprises a hinge region comprising SEQ ID NO:6, 7, 8, 9 or 10.

44. The method of any one of claims 8-42, wherein at least one monomer subunit of the dimer comprises the amino acid sequence of SEQ ID NO:2, SEQ ID NO:3, SEQ ID NO:11, SEQ ID NO:12, SEQ ID NO:13, or SEQ ID NO:14.

45. The method of any one of claims 8-42, wherein at least one monomer subunit of the dimer comprises the amino acid sequence of SEQ ID NO:2.

46. The method of any one of claims 8-42, wherein at least one monomer subunit of the dimer comprises the amino acid sequence of SEQ ID NO:3.

47. The method of any one of claims 8-42, wherein at least one monomer subunit of the dimer is encoded by a polynucleotide comprising the sequence of SEQ ID NO:4.

48. The method of any one of claims 8-42, wherein at least one monomer subunit of the dimer is encoded by a polynucleotide comprising the sequence of SEQ ID NO:5.

49. The method of any one of claims 8-42, wherein the monomer subunits of the dimer each comprises a mutated Factor VIIa (FVIIa) targeting domain conjugated to an immunoglobulin G1 (IgG1) Fc effector domain, wherein the targeting domain comprises the sequence of SEQ ID NO: 17 or SEQ ID NO:18, and wherein the effector domain comprises a hinge region and the effector domain comprises the sequence of SEQ ID NO: 19, SEQ ID NO:20, SEQ ID NO:21, SEQ ID NO:22, SEQ ID NO:23, or SEQ ID NO:24.

50. The method of any one of claims 8-42, wherein at least one monomer subunit of the dimer comprises the amino acid sequence of SEQ ID NO: 11.

51. The method of any one of claims 8-42, wherein at least one monomer subunit of the dimer comprises the amino acid sequence of SEQ ID NO: 12.

52. The method of any one of claims 8-42, wherein at least one monomer subunit of the dimer comprises the amino acid sequence of SEQ ID NO: 13.

53. The method of any one of claims 8-42, wherein at least one monomer subunit of the dimer comprises the amino acid sequence of SEQ ID NO: 14.

54. The method of any one of claims 8-53, wherein the patient substantially maintains his or her vision subsequent to the one or more dosing sessions, as measured by losing fewer than 15 letters in a best-corrected visual acuity (BCVA) measurement, compared to the patient's BCVA measurement prior to the one or more dosing sessions.

55. The method of any one of claims 8-53, wherein the patient exhibits a decrease in the size, volume, and/or thickness of the ocular melanoma subsequent to the one or more dosing sessions, as compared to the size, volume, and/or thickness of the ocular melanoma prior to the one or more dosing sessions.

56. The method of claim 55, wherein the decrease is measured by ultrasound, high-resolution ultrasound biomicroscopy, magnetic resonance imaging, or computed axial tomography.

57. The method of any one of claims 8-56, wherein the patient exhibits a decrease in leakage or blockage of blood vessels in the eye subsequent to the one or more dosing sessions, as compared to the leakage or blockage of blood vessels in the eye prior to the one or more dosing sessions.

58. The method of claim 57, wherein the decrease is measured by fluorescein angiography or indocyanine green angiography.

59. The method of any one of claims 8-58, wherein the patient exhibits a decrease in swelling and/or fluid accumulation beneath the retina or choroid subsequent to the one or more dosing sessions, as compared to the swelling and/or fluid accumulation beneath the retina or choroid prior to the one or more dosing sessions.

60. The method of claim 59, wherein the decrease is measured by ocular coherence tomography.

61. The method of any one of claims 8-60, wherein the patient exhibits a decrease in the size and/or number of iris spots subsequent to the one or more dosing sessions, as compared to the size and/or number of iris spots prior to the one or more dosing sessions.

62. The method of claim 61, wherein the decrease is measured by a gonioscope, slit-lamp biomicroscope, or ophthalmoscope.

63. The method of any one of claims 8-62, wherein the patient experiences an improvement in vision subsequent to the one or more dosing sessions, as measured by gaining 15 letters in a best-corrected visual acuity (BCVA) measurement, compared to the patient's BCVA prior to the multiple dosing sessions.

64. The method of any one of claims 8-63, wherein subsequent to the one or more dosing sessions the neovascularization area is reduced in the eye of the patient, as compared to the neovascularization area prior to the initiation of treatment, as measured by fluorescein angiography or optical coherence tomography.

65. The method of any one of claims 8-64, wherein the patient exhibits a decrease in radiation-induced retinopathy, as compared to radiation-induced retinopathy prior to the one or more dosing sessions.

66. The method of claim 65, wherein the decrease is due to a decreased need for radiation therapy.

67. The method of claim 65, wherein the decrease is measured by fluorescein angiography or indocyanine green angiography.

68. The method of claim 64, wherein the neovascularization area is reduced by at least about 10%, at least about 20%, at least about 30%, at least about 40% or at least about 50%.

69. The method of any one of claims 55-62 and 65-67, wherein the decrease is by at least about 10%, at least about 20%, at least about 30%, at least about 40% or at least about 50%.

70. The method of any one of claims 8-69, wherein subsequent to the one or more dosing sessions, the retinal thickness of the eye of the patient is reduced in the eye of the patient, as compared to the retinal thickness of the eye prior to the initiation of treatment.

71. The method of claim 70, wherein the retinal thickness is reduced by at least about 50 μm, at least about 100 μm, at least about 150 μm, at least about 175 μm, at least about 200 μm, at least about 225 μm, or at least about 250 μm.

72. The method of claim 68, wherein the retinal thickness is reduced by at least about 10%, at least about 20%, at least about 30%, at least about 40%, or at least about 50%.

73. The method of any one of claims 70-72, wherein the decreased retinal thickness is decreased central retinal subfield thickness (CST), decreased center point thickness (CPT), or decreased central foveal thickness (CFT).

74. A method for treating wet age-related macular degeneration (AMD) in an eye of a patient in need thereof, comprising, administering to the patient in multiple dosing sessions, a composition comprising an effective amount of the immunoconjugate dimer of any one of claims 1-2.

75. The method of claim 74, wherein treating the wet AMD comprises preventing, inhibiting or reversing choroidal neovascularization in the eye of the patient in need of treatment.

76. A method for preventing, inhibiting or reversing ocular neovascularization in an eye of a patient in need thereof, comprising, administering to the patient in multiple dosing sessions, a composition comprising an effective amount of the immunoconjugate dimer of any one of claims 1-2.

77. A method for reversing tumor neovascularization in a patient in need thereof, comprising, administering to the patient in multiple dosing sessions, a composition comprising an effective amount of the immunoconjugate dimer of any one of claims 1-2.

78. The method of any one of claims 74-77, wherein the immunoconjugate dimer is a homodimer.

79. The method of any one of claims 74-77, wherein the immunoconjugate dimer is a heterodimer.

80. The method of claim 78 or 79, wherein at least one of the monomer subunits of the immunoconjugate comprises a mutated human FVIIa domain comprising a single point mutation at Lys341 or Ser344.

81. The method of claim 80, wherein the single point mutation is to an Ala residue.

82. The method of claim 81, wherein the single point mutation is Lys341 to Ala341.

83. The method of claim 81, wherein the single point mutation is Ser344 to Ala344.

84. The method of claim 76 and 78-83, wherein the ocular neovascularization is associated with proliferative diabetic retinopathy, wet age-related macular degeneration (AMD), retinopathy of prematurity (ROP), or neovascular glaucoma.

85. The method of any one of claims 76 and 78-83, wherein the ocular neovascularization is secondary to proliferative diabetic retinopathy, wet age-related macular degeneration (AMD), retinopathy of prematurity (ROP), or neovascular glaucoma.

86. The method of any one of claims 76 and 78-83, wherein the ocular neovascularization is choroidal neovascularization.

87. The method of claim 86, wherein the patient has been previously diagnosed with wet age-related macular degeneration (AMD) in the eye.

88. The method of claim 86, wherein the choroidal neovascularization is secondary to wet AMD.

89. The method of claim 87 or 88, wherein the eye of the patient has not been previously treated for choroidal neovascularization or wet AMD.

90. The method of claim 87 or 88, wherein the patient has previously been treated for choroidal vascularization with anti-vascular endothelial growth factor (VEGF) therapy, laser therapy or surgery.

91. The method of any one of claims 74-76 and 78-90, wherein administering comprises intravitreal injection of the composition at each dosing session.

92. The method of any one of claims 74-76 and 78-90, wherein administering comprises suprachoroidal injection of the composition at each dosing session.

93. The method of any one of claims 74-92, wherein the multiple dosing sessions comprise two or more, three or more, four or more or five or more dosing sessions.

94. The method of any one of claims 74-93, wherein each dosing session is spaced apart by from about 20 days to about 50 days, or from about 20 days to about 40 days, or from about 20 days to about 30 days.

95. The method of any one of claim 93 or 94, wherein the multiple dosing sessions comprise 12 to 24 dosing sessions.

96. The method of any one of claims 93-95, wherein administering comprises intravitreal injection of the composition into the eye of the patient once every 28 days, once every 30 days or once every 35 days.

97. The method of any one of claims 74-78 and 80-96, wherein the immunoconjugate comprises the amino acid sequence of SEQ ID NO: 11, 12, 13, or 14.

98. The method of any one of claims 77-83 and 93-97, wherein administering comprises intravenous administration.

99. The method of any one of claims 77-83 and 93-97, wherein administering comprises intratumoral injection.

100. The method of any one of claims 74-76 and 78-97, wherein the patient substantially maintains his or her vision subsequent to the multiple dosing sessions, as measured by losing fewer than 15 letters in a best-corrected visual acuity (BCVA) measurement, compared to the patient's BCVA measurement prior to the multiple dosing sessions.

101. The method of any one of claims 74-76 and 78-97, wherein the patient experiences an improvement in vision subsequent to the multiple dosing sessions, as measured by gaining 15 letters in a best-corrected visual acuity (BCVA) measurement, compared to the patient's BCVA prior to the multiple dosing sessions.

102. The method of any one of claims 74-76, 78-97 and 100-101, wherein subsequent to the multiple dosing sessions, or one or more of the dosing sessions, as measured by fluorescein angiography or optical coherence tomography, the CNV area is reduced in the eye of the patient, as compared to the CNV area prior to the initiation of treatment.

103. The method of claim 46, wherein the CNV area is reduced by at least about 10%, at least about 20%, at least about 30%, at least about 40% or at least about 50%.

104. The method of any one of claims 74-76, 78-97, and 100-103, wherein subsequent to the multiple dosing sessions, or a subset thereof, the retinal thickness of the eye of the patient is reduced in the eye of the patient, as compared to the retinal thickness of the eye prior to the initiation of treatment.

105. The method of claim 103, wherein the retinal thickness is reduced by at least about 50 μm, at least about 100 μm, at least about 150 μm, at least about 175 μm, at least about 200 μm, at least about 225 μm or at least about 250 μm.

106. The method of claim 103, wherein the retinal thickness is reduced by at least about 10%, at least about 20%, at least about 30%, at least about 40% or at least about 50%.

107. The method of any one of claims 104-106, wherein the decreased retinal thickness is decreased central retinal subfield thickness (CST), decreased center point thickness (CPT), or decreased central foveal thickness (CFT).

108. The method of any one of claims 91-97, and 100-107, further comprising measuring the intraocular pressure (IOP) in the eye of the patient prior to each intravitreal or suprachoroidal injection.

109. The method of any one of claims 91-97, and 100-108, further comprising measuring the IOP in the eye of the patient about 20 minutes, about 30 minutes, about 40 minutes, about 50 minutes or about 1 hour after each intravitreal or suprachoroidal injection.

110. The method of claim 108, comprising measuring the IOP in the eye of the patient about 20 minutes, about 30 minutes, about 40 minutes, about 50 minutes or about 1 hour prior to each intravitreal or suprachoroidal injection.

111. The method of any one of claims 108-110, wherein the IOP is measured via tonometry.

112. The method of any one of claims 74-111, further comprises administering an effective amount of a neovascularization inhibitor or an angiogenesis inhibitor to the patient.

113. The method of claim 112, wherein the neovascularization inhibitor or the angiogenesis inhibitor is present in the same composition as the effective amount of the immunoconjugate.

114. The method of claim 112, wherein the neovascularization inhibitor or the angiogenesis inhibitor is present in a different composition than the effective amount of the immunoconjugate.

115. The method of any one of claims 112-114, wherein the neovascularization inhibitor is a vascular endothelial growth factor (VEGF) inhibitor, a VEGF receptor inhibitor, a platelet derived growth factor (PDGF) inhibitor or a PDGF receptor inhibitor.

116. The method of claim 115, wherein the neovascularization inhibitor is ranibizumab.

117. The method of claim 116, wherein the dosage of ranibizumab is from about 0.2 mg to about 1 mg.

118. The method of claim 116 or 117, wherein the dosage of ranibizumab is 0.3 mg or 0.5 mg.

119. The method of any one of claims 80-118, wherein ranibizumab is administered to the eye of the patient via an intravitreal injection.

120. The method of any one of claims 112-119, wherein the composition comprising the effective amount of the neovascularization inhibitor or the angiogenesis inhibitor is administered to the eye of the patient via an intravitreal injection.

121. The method of claim 120, wherein the composition comprising the effective amount of the neovascularization inhibitor is administered at each of the multiple dosing sessions.

122. A pharmaceutical composition comprising the immunoconjugate dimer of any one of claims 1-2.
